SEQUENCE LISTING (8 sequences)

SEQ ID NO: 1 - protein, 19 aa, Moloney murine sarcoma virus
ILNRLVQFVKDRISVVQAL

SEQ ID NO: 2 - protein, 13 aa, Artificial Sequence
  Shortened analogue of melittin peptide
LKVLTTGLPALMS

SEQ ID NO: 3 - protein, 15 aa, Artificial Sequence
  Shortened analogue of melittin peptide
LKVLTTGLPALMSWI

SEQ ID NO: 4 - DNA, 57 bp, Moloney murine sarcoma virus
  misc_feature (1)..(57)
attcttaatcgattagtccaatttgttaaagacaggatatcagtggtccaggctcta

SEQ ID NO: 5 - DNA, 39 bp, Artificial Sequence
  Shortened analogue of melittin peptide
cttaaggtactaactactggactcccagcacttatgtca

SEQ ID NO: 6 - DNA, 45 bp, Artificial Sequence
  Shortened analogue of melittin peptide
cttaaggtactaactactggactcccagcacttatgtcatggatt

SEQ ID NO: 7 - protein, 20 aa, Moloney murine sarcoma virus
CILNRLVQFVKDRISVVQAL

SEQ ID NO: 8 - DNA, 60 bp, Moloney murine sarcoma virus
  misc_feature (1)..(60)
tgcattcttaatcgattagtccaatttgttaaagacaggatatcagtggtccaggctcta

(19) United States
(12) Patent Application Publication
(10) Pub. No.: US 2002/0058020 A1
(43) Pub. Date: May 16, 2002

(54) Isolated amphiphilic peptides derived from the cytoplasmic tail of viral envelope proteins

(76) Inventors: Yanina Rozenberg, Studio City, CA (US); W. French Anderson, San Marino, CA (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/756,250

(22) Filed: Jan. 8, 2001

Related U.S. Application Data
(63) Continuation of application No. PCT/IB99/01261, filed on Jul. 8, 1999.

(51) Int. Cl. A61K 48/00; A01N 63/00; A61K 38/04; C07K 1/00; C07K 5/00; C07K 7/00; C07K 16/00; C07K 17/00; C07K 14/00

(52) U.S. Cl. 424/93.21; 530/350; 530/328

(57) ABSTRACT

An isolated peptide comprising an amino acid sequence derived from a viral envelope protein, wherein at least a portion of the amino acid sequence is located within the cytoplasmic tail or membrane-spanning region of a viral envelope protein. Such peptides are amphiphilic in nature, provide for the destabilization of membranes, and facilitate the entry of viral particles into cells and the efficient formation of viral particles. The peptides may, in another embodiment, be attached to the viral membrane, along with a targeting polypeptide, as part of an artificial viral envelope protein.

This is a continuation of International Application No. PCT/IB99/01261, filed Jul. 8, 1999, the contents of which are incorporated herein by reference.

This invention relates to isolated amphiphilic peptides that are derived from the cytoplasmic tail and/or the membrane-spanning region of viral envelope proteins and, in particular, the cytoplasmic tail region of the transmembrane subunit of retroviral envelope proteins, and to derivatives or analogues of such peptides that maintain the amphiphilic structure of such peptides, which provides their membrane destabilization activity.

This invention also relates to modified enveloped viruses in which the viral envelope is modified to include the foregoing peptides or their derivatives or analogues. For example, the present invention relates to retroviruses having modified envelope proteins, wherein the peptides or their derivatives or analogues are included in the surface (SU) subunit and/or the external region of the transmembrane (TM) subunit of the retroviral envelope protein, or are attached to the exterior and/or interior of the retroviral membrane, independently of and in addition to, or in lieu of, the viral envelope protein. Such modified enveloped viruses also may include a targeting peptide containing a binding region that binds to a ligand.

### BACKGROUND OF THE INVENTION

Retroviruses in general include a “core” that contains a retro-viral genome, nucleoprotein, protease, reverse transcriptase, and integrase enclosed within a capsid. A retroviral envelope surrounds the capsid. The retroviral envelope includes a viral membrane and viral envelope protein. The retroviral envelope protein is a post-translationally cleaved heterodimer of a surface subunit (SU) and a transmembrane subunit (TM). The TM includes an external region which is on the external side of the viral membrane and is complexed or associated with the SU; a membrane-spanning region, which is located within the viral membrane; and a cytoplasmic tail region, which is on the internal side of the viral membrane.

The retroviral envelope protein (Env) is functional at two key steps in host or target cell entry: 1) binding of the cellular receptor and 2) fusion with the cellular membrane. The initial steps in viral entry are understood in considerable detail (White, Science, Vol. 258, pgs. 917-924 (1992)). The first interaction between a retrovirus and a host or target cell occurs as the SU binds to a receptor on the cell. Subsequent to such binding, the TM undergoes a major conformational change during which its N-terminal end, known as the “fusion peptide,” is liberated from its hydrophobic environment within the SU and inserts into the host or target cell membrane. Peptides representing the portion of the TM immediately adjacent to the fusion peptide have the propensity to separate into monomers within the host cell membrane. (Yu, et. al., Science, Vol., 266, pgs. 274-276 (1994)). Such an event may initiate juxtaposition of the viral and host or target cell membranes.

The viral envelope protein-cell receptor interaction is followed by multiple receptor recruitment, which is speculated to assist in the merging of the two membranes (Melikyan, et al., J. Cell. Biol., Vol. 13, pgs. 679-691 (1995)). The current literature provides evidence that, whereas a viral envelope protein is necessary and sufficient to induce full fusion (Jones, et al., J. Virol., Vol, 67 pgs. 67-74 (1993)), an envelope protein ectodomain (i.e., the external region) attached to a membrane by a glycolipid linker anchor induces fusion of only the outer lipid bilayers (i.e., hemifusion), but does not cause complete fusion (Kemble, et al., Cell, Vol.76, pgs. 383-391 (1994)). Such data provide speculation that the membrane-spanning region and/or cytoplasmic tail region of the TM may be required for bringing envelope protein mediated fusion to completion. Thus, the present invention is directed to a cytoplasmic tail domain or region of the envelope protein that may lower the kinetic barrier to membrane fusion.

### SUMMARY OF THE INVENTION

The present invention is directed to isolated peptides including an amino acid sequence having an amphiphilic structure. The amino acid sequence may be derived from a viral envelope protein and, in particular, a retroviral envelope protein. Such amino acid sequence includes at least a portion of the amino acid sequence present in the cytoplasmic tail region of the TM of the envelope protein adjacent to the membrane-spanning region of the TM of the envelope protein. The peptide may or may not include at least a portion of the membrane-spanning region of the TM. These peptides provide for the destabilization of membranes and facilitate the efficient formation of viral particles. Accordingly, an isolated peptide comprising a fragment of a viral envelope protein, wherein said peptide is free of the portion of the envelope protein N-terminal of the membrane-spanning region of the envelope protein is provided by the present invention, said peptide having a membrane-destabilizing activity.

The present invention also is directed to viral vectors or viral particles (virions) wherein the envelope protein of the virus is modified to include one or more peptides, which peptide(s) have the propensity to form amphiphilic structures, particularly amphiphilic alpha-helical structures, and may be derived from a viral envelope protein or which may be obtained from other sources, and wherein the peptide(s) is incorporated into a portion of the envelope protein that is exterior to the viral membrane. Such modified envelope proteins also may include a targeting polypeptide containing a binding region that binds to a ligand or the targeting polypeptide may be attached separately to the viral membrane. The peptide of the present invention aids in host or target cell entry by providing an additional membrane-active component for fusing the viral vectors or vector particles to such cells.

Alternatively, the peptides of the present invention may be attached to the viral membrane of the viral vector or viral particle and such vector or viral particle may or may not include an envelope protein. In the case of the alternative embodiment in which the viral vector or viral particle includes an envelope protein, the peptide is attached separately to the viral membrane and is not incorporated into the envelope protein. The envelope protein may be a wild type viral envelope protein, or may be a modified viral envelope protein including a targeting polypeptide. In the case in which the viral vector or viral particle does not include an envelope protein, the peptide(s) of the present invention form an “artificial envelope protein.” In one embodiment, the “artificial envelope protein” also includes a targeting polypeptide.

The present invention also is directed to packaging cells and producer cells that include polynucleotides encoding the peptides of the present invention. Such packaging cells and producer cells generate modified viral vectors or viral particles as hereinabove described that include the peptides as a portion of the viral envelope protein or in which the peptides are separately attached to the exterior and/or interior of the viral membrane.

Thus, in accordance with an aspect of the present invention, a viral particle to be used as a viral vector is provided with an amphiphilic peptide on the outer surface thereof and such viral particle may or may not include a wild type envelope protein. In the case in which the viral particle includes an envelope protein, the amphiphilic polypeptide may be incorporated into the envelope protein or may be attached to the viral membrane as an entity separate from the viral envelope protein. In the case in which the viral particle does not include an envelope protein, the amphiphilic peptide is attached to the viral membrane as part of an “artificial envelope protein.”

### DEFINITIONS

In accordance with the present invention and as used herein, the following terms are defined with the following meanings, unless used explicitly otherwise.

The term “amino acid,” as used herein, means both natural and unnatural amino acids in either the L- or D- forms. Natural amino acids are those found in nature (Morrison and Boyd, Organic Chemistry, 4th edition, pgs. 1118-1119 (1983)). Unnatural amino acids are those not found in nature but capable of being synthesized and include, but are not limited to norleucine, norvaline, and ornithine.

The term “amphiphilic,” as used herein, means that a peptide or other molecule contains both hydrophobic and hydrophilic regions. An amphiphilic peptide or other molecule may have a structure such that one side is hydrophobic and the other side is hydrophilic. The amphiphilicity of a structure within the meaning of the present invention may in particular be characterized by its hydrophbic moment μ.

The term “polynucleotide,” as used herein, means a polymeric form of nucleotide of any length, and includes ribonucleotides and deoxyribocleotides. This term also includes single- and double-stranded DNA, as well as single- and double-stranded RNA. In addition, the term includes modified polynucleotides, such as methylated or capped polynucleotides.

The term “polypeptide,” as used herein, means a polymer of amino acids and does not refer to any particular length of polymer. Such term also includes post-translationally modified polypeptides or proteins (e.g., glycosylated, acetylated, phosphorylated, etc.).

The term “ligand,” as used herein, means a molecule that is capable of being bound by a targeting polypeptide. Such molecules include, but are not limited to, cellular receptors and extracellular components such as extracellular matrix components.

### BRIEF DESCRIPTION OF THE DRAWINGS

The invention now will be described with respect to the drawings, wherein:

FIG. 1 shows schematic representations of MoMuLV Env and its membrane-proximal region. A. Diagram of MoMuLV Env surface subunit (SU or gp70) and transmembrane subunit (TM or p15E). The membrane-proximal region of the envelope protein, i.e., amino acid residues 598-616, is shown in stripes with the corresponding amino acid sequence (shown in single letter code). B. The amphiphilic character of region 598-616 as predicted by the Schiffer and Edmunson's helical-wheel method (generated by DNASIS, Hitachi software). C. Ribbon diagram of the region 598-616 modeled as an α-helix. In the figure the hydrophilic amino acids are represented in black and the hydrophobic ones in white.

FIG. 2 provides graphs which show that peptide 598-616 of Moloney Murine Leukemia Virus forms an amphiphilic α-helix in the presence of membranes.

A. Membrane-Dependent Formation Of An α-Helical Structure By Peptide 598-616 As measured By Circular Dichroism (CD). The plot represents an average of 9 CD spectra for 50 μM peptide 598-616 in an aqueous solution (20 mM NaPO₄,pH 7, dashed line), and in presence of liposomal membrane vesicles (POPG/POPC [1:3] liposomes in 20 mM Na₂ PO₄, pH 7.4; lipid to peptide molar ratio 100:1; solid line). The measurements were obtained for wavelength 195-250 nm at 20 mdeg sensitivity. The y-axis shows the ellipticity(t) x10⁻⁴ (deg cm² dmol⁻¹). The x-axis shows the wavelength (nm).

B. Spin-Labeled Peptide 598-616 Associates With Membranes As Determined By Electron Paramagnetic Resonance (EPR) Analysis. The characteristic EPR spectrum for nitroxide-labeled peptide 598-616 V606C(R1) in an aqueous environment (dashed line) and in the presence of liposomes (POPG/POPC at 1:3 molar ratio in PBS, pH7.4; lipid to peptide molar ratio 100:1; solid line). The arrow marks the position of a small component of free spin.

C. Peptide 598-616 Forms A Membrane-Associated Amphiphilic α-Helix As Determined By EPR. The parameter φ was derived for the peptide 598-616 with a single residue (X-axis) substituted by Cys and spin-labeled for analysis with the nitroxide The interface between the lipid and aqueous phases is between phi=0 and phi=1.

FIG. 3 shows induction of current flux induction across a planar membrane by the wild type or native peptides 598-616, 617-632, or a mutant peptide 598-616 R609C (final peptide concentration 3 μM; POPG/POPC 1:3; black membrane 500 microns in diameter was kept at +50 mV in 100 M KCl, 10 mM Tris-HCl pH 7.5). The horizontal arrow indicates membrane rupture. The insert demonstrates interaction of bacterial porin (5 ng/ml) with the planar membrane. The y-axis shows the membrane current (pA). The x-axis shows the time (min).

FIG. 4 shows the effect of mutations in the Env cytoplasmic tail region on Env ability to induce cell-to-cell fusion. A. Relative Env fusion and viral titers for Env with cytoplasmic tail region truncations. Viral particles were obtained from 293T cells transfected transiently (n=3−5) with the expression plasmids for env, gag-pol, and β-gal, with wild type or mutant (616*, 601*, 598*, 595SR*, 578*, GLA ecto) env. Transduction efficiency of these virions (white bars=titers) was tested on NIH3T3 cells. The Env fusogenicity (filled bars =% fusion) was measured by expressing Env only in 293T cells. The indicator XC6 cells were added 24 hours post-transfection, were fixed 12 hours later and scored microscopically on ten 2 mm² grids for syncytia (cells with four or more nuclei). The left y-axis indicates the titers. The right y-axis indicates the fusion in % cell to cell fusion.

B. Rate Of Fusion By Env With Cytoplasmic Mutations. Ecotropic receptor-expressing 293/12 cells transiently transfected with wild type or mutant (616*, 601*, 595SR*, GLA ecto) Envs were scored for syncytia at the indicated post-transfection time. The average number of syncytia per 2 mm² grids (n=10) is plotted (y-axis). The x-axis indicates the time (hrs). The data shown are from a representative experiment.

C. Syncytia Formation By Env With Cytoplasmic Substitutions. NIH3T3 cells were photographed 24 hours post-transfection with the R-less Env constructs containing wild type membrane-proximal region 598-616 (picture no. I), or substituted with the melittin fragment (picture no. II), the hydrophilic (picture no. III), or the random sequences (picture no. IV).

FIG. 5 shows the efficiency of particle incorporation for Env mutants with truncations (A), or point mutations (B), or substitutions (C) in cytoplasmic tail region. Supernatant from 293T culture transiently transfected with the three expression plasimids encoding the env, gag-pol, and β-gal genes was the source of virions used for Western Blot analysis with anti- gp 70, anti- p 30, and/or anti- p15 E antibodies. The mutant Env used in transfections are indicated above the gels. H₂O-mock transfected. In panel (A) the top half of the gel was exposed only to the anti −gp70 antibody and the bottom to the anti- gp30 and anti −p15E antibodies.

FIG. 6 shows a hypothetical model of the MoMuLV envelope protein sub-ectodomain region (i.e., the memberane-spinning region and the cytoplasimic tail region). A. The monomer of the submembrane (i.e., the cytoplasimic tail region) envelope protein segment before and after the R peptide cleavage as described herein. The position of Arg 609 is shaded, hydrophobic regions are in white. B. The proposed sub-ectodomain unit shown as a trimer of two unprocessed tails and one R-less tail. C. HIV-1 matrix trimer as crystallized by Hill, et al., 1995. The representation is based on the coordinates obtained from the Brookhaven web site, Accession No. 1 HIW.

### DETAILED DESCRIPTION OF THE INVENTION

In accordance with an aspect of the present invention, there is provided an isolated peptide. The peptide, in one embodiment, comprises an amino acid sequence derived or isolated from a viral envelope protein, wherein such peptide forms an amphiphilic structure. The peptide is derived from a viral envelope protein amino acid sequence, at least a portion of which is located in the cytoplasmic tail region and adjacent to the membrane-spanning region of the transmembrane subunit of the envelope protein. In general, such peptide comprises a fragment of a viral envelope protein which is free of the SU and the external region of the TM, or all sequences 5′ of the membrane spanning region of the transmembrane subunit. In one embodiment, this fragment includes at least the first four amino acids of the N-terminal portion of the cytoplasmic tail region of the transmembrane subunit of the envelope protein. Preferred is an embodiment which includes at least the first six amino acids of the N-terminal portion of the cytoplasmic tail region of the transmembrane subunit of the envelope protein. More preferred is an embodiment including the first eight amino acids.

In another embodiment, the peptide comprises an amino acid sequence that is; a derivative or analogue of the amino acid sequence hereinabove described. The derivative or analogue may have at least one substitution of an amino acid residue of the abovementioned amino acid sequence. In another embodiment, the analogue may include a reverse amino acid sequence as compared to the amino acid sequence present in the env protein from which it is derived. The derivative or analogue may either include D or L amino acids.

Also within the scope of the present invention are analogues of the peptides of the invention that employ other backbones than peptidic backbones and retain the overall stereochemical positions of the R-groups of the peptides. An example of such a backbone is a peptide-amide backbone. Polynucleotides may also provide such a backbone, e.g. a methylphosphonate backbone may serve as the backbone structure carrying the relevant R-groups.

In one embodiment, the peptide is comprised of an amino acid sequence derived from the cytoplasmic tail region of the TM as hereinabove described, and further includes at least a portion of the membrane-spanning region of the TM.

The isolated peptides of the present invention are amphiphilic peptides, which may have an alpha-helical secondary structure, especially in the presence of membranes. Alternatively, the isolated amphiphilic peptides may have a different secondary structure, such as a beta sheet.

The peptides, derivatives and analogues of peptides contemplated in the present invention have membrane destabilizing activity. Various methods of determining the membrane destabilizing activity of a compound are known to the person skilled in the art and may be employed to determine the membrane destabilizing activity within the meaning of the present invention. In particular, electrophysiological methods of determining the membrane destabilizing activity may be used. Such methods include measuring the release of a suitable marker, such as e.g. a cation, such as e.g. potassium, from a liposome under defined conditions (see e.g. example 1). Release of a suitable fluorescent marker from a liposome may also be measured in a suitable assay.

An alternative assay is the planar lipid bilayer integrity assay as known in the art and described in example 1.

Accordingly, peptides wherein the membrane-destabilizing activity of said peptide is sufficient to induce an detectable increase of the release of a suitable marker from a liposome are part of this invention. In particular, such a detectable increase will occur at an active concentration of 30 mM peptide/1 mol lipid in a suitable electrophysiological assay. Preferred is a peptide that will show such an increase at an active concentration of 10 mm peptide/1 mol lipid. Most preferred is a peptide that will show such an increase at: an active concentration of 0.5 mM peptide/1 mol lipid.

In a preferred embodiment, the potassium release from a liposome as measured in a potassium release assay as essentially described in example 1 (for POPC:POPG, 1:1; see Table 3), will be more than 10%. In a more preferred embodiment it will be more than 20% under the same conditions.

The preferred peptides of the invention have an amphiphilic structure, in particular an a-helical amphiphilic structure. In a preferred embodiment, the peptides, derivatives and analogues of peptides contemplated in the present invention have an amphiphilic structure with a hydrophobic moment of at least 0.9 as calculated using the DNASIS software employing the Chou, Fasman and Rose algorithm and calculated with the Kyte and Doolittle algorithm. Preferred is a hydrophobic moment of at least 1.0.

The present invention also contemplates the use of an amphiphilic compound other than a peptide, derivative or analogue of a peptide having a membrane-destabilizing activity for the preparation of a viral vector.

In particular, compounds wherein the membrane-destabilizing activity of said compound is sufficient to induce a detectable increase of the release of a suitable marker from a liposome are part of this invention. In a preferred embodiment, such a detectable increase will occur at an active concentration of 30 mM compound/1 mol lipid in a suitable electrophysiological assay. Preferred is a compound that will show such an increase at an active concentration of 10 mM compound/1 mol lipid. Most preferred is a compound that will show such an increase at an active concentration of 0.5 mM compound/1 mol lipid.

The preferred compounds of the invention have an amphiphilic structure. In a preferred embodiment, the compounds contemplated in the present invention have an amphiphilic structure with a hydrophobic moment of at least 0.9 as calculated using the DNASIS software employing the Chou, Fasman and Rose algorithm and calculated with the Kyte and Doolittle algorithm. Preferred is a hydrophobic moment of at least 1.0.

In general, the isolated peptides of the present invention can be of various lengths. In one embodiment, the peptides include an amphiphilic amino acid sequence having from 8 to 40 amino acid residues, in a preferred embodiment the peptides of the invention include 12 to 35 amino acid residues. In a particular preferred embodiment the peptides include at least 8 amino acid residues. Such isolated peptides, which include amino acid residues that are derived from the cytoplasmic tail region of the TM, and may also include amino acid residue(s) from the membrane-spanning region of the TM, also sometimes are hereinafter referred to as “membrane-proximal” amphiphilic peptides. Representative examples of such peptides are given in Table I below. For purposes of this application, negative numbers refer to the number of amino acid residues in the C-terminal portion of the predicted membrane-spanning region of the transmembrane subunit of the viral envelope protein, and positive numbers refer to the number of amino acid residues from the cytoplasmic tail region of the TM, beginning at the N-terminal residue of the tail, that are in the peptide. The boundary between the membrane-spanning region of the transmembrane subunit and the cytoplasmic tail region of the TM is at the first hydrophilic amino acid residue after the stretch of about 20 hydrophobic amino acids C-terminal to the external region of the transmembrane subunit. Thus, for example, a peptide denoted as “−2/14” means that the isolated peptide includes (in an N-terminal to C-terminal direction), as the first two amino acid residues of the N- terminus, the two C-terminal amino acids of the predicted membrane-spanning region of the transmembrane subunit and, as the last 14 amino acid residues, the 14 N-terminal amino acids of the predicted cytoplasmic tail region. Peptides denoted by two positive numbers are peptides that are contained only in the cytoplasmic tail region of the viral envelope protein. Thus, for example, a peptide denoted as “1/17” means that the isolated peptide includes the 17 N-terminal amino acids of the predicted cytoplasmic tail region. Table I lists the positions of the most amphiphilic membrane-proximal segments in a number of viral envelope proteins. The scope of the present invention, however, is not intended to be limited thereby. Table I also does not imply the most active size or residue identity of the segments listed therein. Also, in the membrane-proximal domains of surface viral proteins other than viral envelope proteins, analogous amphiphilic regions are detected, such as, for example, in the M2 protein of influenza virus, and in the spike protein of adenovirus. Non-viral lytic peptides also contain stretches of similar characteristics (e.g., melittin). An artificial or synthetic amphiphilic sequence can be generated to mimic the amphiphilic membrane-destabilizing properties of the wild-type peptides identified herein as illustrated by the use of a melittin analogue described below. The following abbreviations are used in Table I: ALV-avian leukosis virus; BLV-bovine leukemia virus; EIA-equine infectious anemia; FIV-feline immunodeficiency virus; HEP C-hepatitis C; HIV-human immunodeficiency virus; HTLV-human T-cell leukemia virus; hRSV-human respiratory syncytial virus; infM2-influenza M2virus; INF-influenza; MMTV-Mouse Mammary Tumor Virus; MPMV-Mason Pfizer monkey virus; RSV-Rous Sarcoma Virus; PINF-parainfluenza; SNV-spleen necrosis virus; VSV-vesicular stomatitis virus; SimSrcV-HLB-simian sarcoma virus; MoMuLV-Moloney Murine Leukemia Virus.

TABLE I

| VIRUS | SEGMENT |
|---|---|
| ALV | −2/14 |
| BLV | −2/17 |
| EIA | 1/52 |
| FIV | −6/10 |
| HEP C | 1/17 |
| HIV 1 | −3/11 |
| HIV 25YR | 1/25 |
| HTLV2 | 1/12 |
| HRSV | 1/21 |
| InfM2 | 1/16 |
| INFAl | −2/11 |
| MoMuLV | −3/14 |
| MMTV | −6/13 |
| MPMV | 1/22 |
| RSV | −9/8 |
| PINF | 1/17 |
| SIV239 | −5/13 |
| SNV | −2/16 |
| VSV | −2/13 |
| SimSrcV-HLB | −9/8 |

In general, such peptides include at least one hydrophilic amino acid residue which is “out-of-phase” (i.e., a hydrophilic amino acid residue in a hydrophobic region of the predicted amphiphilic structure). Although Applicants do not intend to be limited by any theoretical reasoning, it is believed that, when such peptides are contained in the cytoplasmic tail region of a viral envelope protein, they enter a cell membrane at an oblique angle. A structural distortion resulting from an out-of-phase amino acid residue may be involved in providing an oblique angle needed for membrane destabilization during fusion (Martin, et al., J. Virol. Vol. 70, pgs. 298-304). The resulting changes in membrane curvature thus may decrease the energy required for fusion of lipid bilayers. This mechanism may be employed and thus preserved evolutionarily by viruses in order to potentiate efficient fusion with a host cell.

In one embodiment, the peptide has the amino acid sequence (SEQ ID NO: 1), which is as follows:

ILNRLVQFVKDRISVVQAL

This peptide corresponds to amino acid residues 598-616 of the wild type envelope protein of Moloney Murine Leukemia Virus. The residues ILN are from the predicted membrane-spanning region of the transmembrane subunit, whereas the residues RLVQFVKDRISVVQAL are from the predicted cytoplasmic tail region of the TM. In another embodiment, the peptide is an analogue or derivative of (SEQ ID NO: 1) which has at least one substitution of (SEQ ID NO: 1) that maintains the amphiphilic structure and membrane destabilization activity of the peptide.

Such peptides may be employed in providing viral vectors that include the peptides as part of a modified envelope protein, or wherein the peptides are attached separately to the exterior and/or interior of the viral membrane. When the peptides are attached separately to the exterior and/or interior of the viral membrane, the viral vector may or may not include a viral envelope protein. When the viral vector does not include a viral envelope protein, the peptides are part of an “artificial envelope protein.”

Alternatively, the peptide that is included in the modified viral envelope protein or is attached to a viral membrane as hereinabove described is a synthetic peptide or a naturally occurring peptide which is obtained from an organism other than a virus, which peptide is a biologically active amphiphilic peptide, such as, for example, melittin peptide, magainin peptides, XPF peptides, PGLa peptides, CPF peptide, and defensins. In a preferred embodiment, the peptide is an analogue, fragment, or derivative of melittin peptide. In particular, such peptide has the following structural formula:

LKVLTTGLPAL (X) S (W)ₘ(I)ₙ,

wherein X is isoleucine or methionine, m is 0 or 1, and n is 0 or 1. In one embodiment, X is methionine, each of m and n is 0, and the peptide has the following structure:

LKVLTTGLPALMS. (SEQ ID NO: 2).

In another embodiment, m is 1, n is 1, X is methionine, and the peptide has the following structure:

LKVLTTGLPALMSWI. (SEQ ID NO: 3).

In another embodiment, the peptide is an analogue or derivative of (SEQ ID NO: 2) or (SEQ ID NO: 3) which may have at least one substitution that maintains the amphiphilic or alphahelical structure and the general functional properties of the peptide. In yet another embodiment, the analogue may include a reverse amino acid sequence as compared to the amino acid sequence present in the env protein from which it is derived. The derivative or analogue may either include D or L amino acids.

Thus, in accordance with one embodiment of the present invention, amphiphilic peptides that preferably form an alpha-helical structure are used for producing an artificial envelope protein or for modifying an existing envelope protein of a viral vector.

In accordance with another aspect of the present invention, there is provided an enveloped virus wherein the viral envelope is modified to include the amphiphilic peptide hereinabove described at one or more locations of the exterior portion of the viral envelope. The amphiphilic peptide aids in fusing the virus to cells. Preferably, the modified viral envelope further includes a targeting polypeptide containing a binding region that binds to a ligand.

Enveloped viruses that may include the amphiphilic peptide, and a targeting polypeptide, if desired, in the viral envelope include, but are not limited to, enveloped RNA viruses and enveloped DNA viruses. Enveloped RNA viruses include, but are not limited to, retroviruses (including murine leukemia viruses and gibbon ape leukemia virus); alphaviruses (including Sindbis virus); arenaviruses; orthomyxoviruses; paramyxoviruses; and coronaviruses. Enveloped DNA viruses include, but are not limited to, Herpes viruses (including Herpes Simplex Virus) and poxviruses. In such viruses, the isolated peptides of the present inventon are derived from the cytoplasmic tail region of the viral envelope protein and may or may not include amino acids derived from the membrane-spanning region of the viral envelope protein.

In one embodiment, the enveloped virus is a retrovirus.

In yet another embodiment, the amphiphilic peptide is incorporated into the envelope protein in a region that is neither the cytoplasmic tail region nor the membrane-spanning region of the transmembrane subunit. The amphiphilic peptide may be located in any position in the envelope protein that is suitable for presenting the peptide in a functional manner. In one embodiment, the peptide is placed at the N-terminal end of the surface subunit of the envelope protein. In another embodiment, when the envelope protein is a Moloney Murine Leukemia Virus envelope protein, the peptide may be placed between amino acid residues 6 and 7 of the receptor binding region or at the N-terminus BstI site located between residues 16 and 17 of the receptor binding region. The peptide also may be inserted into or substituted for conserved exposed cysteine-constrained loops of the envelope protein (e.g. in the region of residues 74-84, or 177-181) of the receptor binding region. In one embodiment the exposed loops as recently identified based on the crystallographic resolution of the tropism-determining segment from the Friend Murine Leukemia Virus (Fass, et al., Science Vol. 277, Pgs.1662-1666,(1997) may be useful for the insertion of the functional peptides into the envelope protein. The examples of such locations in Moloney envelope protein nomenclature are the exposed loop 1 (residues 90-93), the exposed loop 2 (residues 111-114), the exposed loop 3 (residues 121-126) or the exposed loop 4 (residues 210-216).

The peptide also may be inserted into the hypervariable polyproline or “hinge” region of the envelope protein. In one embodiment, amino acid residues 34 through 49 of the hypervariable polyproline region of the Moloney Murine Leukemia Virus envelope protein are removed and replaced with a peptide as hereinabove described. In another embodiment, the peptide is inserted between amino acid residues 35 and 36 of the hypervariable polyproline region the Moloney Murine Leukemia Virus envelope protein. These locations are provided as examples and are not intended to be either the exact or the limiting possibilities for the insertion of the functional peptides into the SU of Moloney Murine Leukemia Virus. In yet another embodiment, the amphiphilic peptide may precede the first N-terminal residue of the SU. In yet a further embodiment, the amphiphilic peptide may be after the last C-terminal residue of the SU.

In one embodiment, there is provided a polynucleotide encoding a modified envelope protein which includes the amphiphilic peptide hereinabove described, wherein the amphiphilic peptide, in addition to being present in the cytoplasmic tail region of the TM, also is present in an external portion of the envelope protein at one or more positions. The modified envelope protein also may include a targeting polypeptide, as hereinabove described. Such a polynucleotide may be constructed in accordance with genetic engineering techniques known to those skilled in the art.

In one embodiment, when the amphiphilic peptide has the sequence (SEQ ID NO: 1), the polynucleotide encoding the modified envelope protein includes the nucleic acid sequence (SEQ ID NO: 4), or a degenerate sequence thereof.

In another embodiment, when the amphiphilic peptide has the sequence (SEQ ID NO: 2), the polynucleotide encoding the modified envelope protein includes the nucleic acid sequence (SEQ ID NO: 5), or a degenerate sequence thereof.

In yet another embodiment, when the amphiphilic peptide has the sequence (SEQ ID NO: 3), the polynucleotide encoding the modified envelope protein includes the nucleic acid sequence (SEQ ID NO: 6), or a degenerate sequence thereof.

Such a polynucleotide as hereinabove described may be employed in the generation of the viral vectors or viral particles described hereinabove. Such viral vectors or viral particles of the present invention may be constructed by a variety of methods known to those skilled in the art.

For example, such viral vectors or viral particles may be generated from packaging cells and producer cells that include polynucleotides encoding the retroviral gag and pol proteins, and one or more polynucleotides that encode the components of the modified viral envelope proteins hereinabove described.

The polynucleotide encoding the modified envelope protein, which includes the amphiphilic peptide, may be contained in an appropriate expression vehicle, such as a retroviral expression plasmid, such as those further described herein, which is transfected into an appropriate “pre-packaging” cell line that includes nucleic acid sequences encoding the retroviral gag and pol proteins, whereby the “pre-packaging” cell line becomes a packaging cell line. Examples of “pre-packaging” cell lines that may be transfected with the polynucleotide encoding the modified envelope protein, include GP8 cells, GPL cells, and GPNZ cells as described in Morgan, et al., J. Virol., Vol. 67, No. 8, pgs. 4712-4721 (August 1993).

The polynucleotide may be transfected into the pre-packaging cells through any means known in the art. Such means include, but are not limited to, electroporation, the use of liposomes, and CaPO₄ precipitation. The resulting packaging cells may be transfected with an appropriate retroviral expression plasmid, such as those described herein, and that may include a polynucleotide encoding a therapeutic agent by means known to those skilled in the art, to form a producer cell line. Such producer cells generate infectious retroviral vector particles that include the modified envelope protein hereinabove described, in which the amphiphilic peptide is located in the external portion of the viral envelope protein as well as in the cytoplasmic tail region.

In another embodiment, a polynucleotide encoding a modified envelope protein that includes the amphiphilic peptide and the targeting polypeptide, is contained in an appropriate expression vehicle, and is transfected into an appropriate pre-packaging cell line as hereinabove described to form a packaging cell. The packaging cell then may be transfected with an appropriate expression vehicle such as those described herein to form a producer cell, which generates infectious retroviral particles that include a modified envelope protein that includes the amphiphilic peptide and the targeting polypeptide in the external portion of the retroviral envelope protein.

In another embodiment, there is provided a retroviral particle that includes a retroviral envelope protein. The retroviral envelope protein may be an unmodified wild type retroviral envelope protein, or may be a modified retroviral envelope protein that includes a targeting polypeptide, wherein a portion of the viral envelope protein is replaced with a targeting polypeptide. The retroviral particle also includes the amphiphilic peptide, which is attached separately to the viral membrane. In one embodiment, the amphiphilic peptide is attached separately to the viral membrane via an anchor comprised of at least a portion of a membrane-spanning region of a viral envelope protein such as, for example, the membrane-spanning region of the TM of a retroviral envelope protein. In another embodiment, the amphiphilic peptide is attached separately to the viral membrane by chemical means, such as those described below.

In one embodiment, such a retroviral particle may be generated by transfecting a pre-packaging cell line with a first polynucleotide and a second polynucleotide. The first polynucleotide encodes an unmodified wild type retroviral envelope protein or a modified viral envelope protein that includes a targeting polypeptide as hereinabove described. Such polynucleotide may be contained in a retroviral expression plasmid. The second polynucleotide includes a nucleic acid sequence encoding the amphiphilic peptide hereinabove described, and a nucleic acid sequence encoding at least a portion, and in one embodiment, all, of the membrane-spanning region of the transmembrane subunit of a viral envelope protein with or without nucleic acid sequences encoding the cytoplasmic tail region of the TM. In one embodiment, the nucleic acid sequence encoding the membrane-spanning region of the transmembrane subunit is located 5′ to the nucleic acid sequence encoding the amphiphilic peptide. In another embodiment, the nucleic acid sequence encoding the membrane-spanning region of the transmembrane subunit is located 3′ to the nucleic acid sequence encoding the amphiphilic peptide.

Upon transfection of a pre-packaging cell with the first and second polynucleotides, a packaging cell line is formed. A producer cell line then may be formed from the packaging cell line by means known to those skilled in the art. The resulting producer cell line generates viral particles that include the modified envelope protein including the targeting polypeptide. The amphiphilic peptide is attached to the viral membrane as an entity separate from the viral envelope protein, on either the exterior or the interior of the viral membrane or on both sides of the viral membrane.

Alternatively, a viral vector or viral particle including a modified envelope protein, including a targeting polypeptide, may be generated from a pre-packaging cell as hereinabove described. The amphiphilic peptide then is attached to the viral membrane by chemical means.

For example, in one embodiment, the amphiphilic peptide of the present invention may be attached to the viral membrane first by forming a peptide-lipid conjugate. Such a conjugate may be formed by ligating a lipid such as, for example, a lipid having a maleimidoyl moiety, to an amino group in the peptide. The conjugate may be prepared according to the standard protocols in an aprotic solvent. After the reaction is completed, preliminary purification may be achieved by gel filtration on Sephadex LH-20 in dimethylformamide followed by precipitation of the conjugate with ether. The purity of the conjugate then is verified by mass spectrometry.

The attachment of the conjugate to the viral membrane is carried out by mixing small quantities of the conjugate, dissolved in acetonitrile, with the viral particles. Preferably, the amount of conjugate should not exceed 10 to 15% of the total amount of lipid in the resulting modified viral envelope. The viral particles now including the amphiphilic peptide attached to the viral membrane may be purified by means known to those skilled in the art.

Alternatively, a lipid-polyethylene glycol (PEG)—amphiphilic peptide conjugate may be attached to the viral membrane. For example, a lipid-peptide conjugate such as hereinabove described may be attached to a polyethylene glycol polymer having a molecular weight of about 2,000 and bearing a distal sulfhydryl group, to form a lipid—PEG—peptide conjugate. The conjugate then can be purified by employing gel-filtration chromatrography in an aprotic medium (e.g., Sephadex LH-20 in DMF), or by employing gel filtration/absorption chromatography on a Toyopearl HW-40 (Toyo Soda, Japan) in DMF, tetrahydrofuran, or methanol.

The lipid—PEG—peptide conjugate may be attached to the viral membrane by mixing a small quantity of a solution of the lipid—PEG—peptide conjugate in acetonitrile with the viral particles. The resulting viral particles thus have the amphiphilic peptide attached to the viral membrane. Because the resulting viral particles also include polyethylene glycol, the resulting viral particles also will be less likely to be recognized by the immune system.

In another embodiment, there is provided a retroviral vector particle which includes a naturally occurring or wild-type or native, retroviral envelope protein. Such retroviral vector particle also includes the amphiphilic peptide and the targeting polypeptide hereinabove described, wherein the amphiphilic peptide and the targeting polypeptide are attached to the viral membrane. In one embodiment, each of the amphiphilic peptide and the targeting polypeptide is attached individually to the viral membrane. Such attachment may be through an anchor comprised of at least a portion of the membrane-spanning region of a transmembrane subunit of a viral envelope protein, or through a glycolipid linker, or through a peptide—lipid conjugate as hereinabove described. In another embodiment, a polypeptide is formed which includes the targeting polypeptide, the amphiphilic peptide, and a spacer moiety, such as, for example, a Glycine-Serine-Glycine tripeptide placed between the targeting polypeptide and the amphiphilic peptide. The resulting polypeptide is attached to the viral membrane. Such attachment may be accomplished via at least a portion of the membrane-spanning region of a transmembrane subunit, a glycolipid linker, or through a peptide-lipid conjugate as hereinabove described.

In one embodiment, when each of the amphiphilic peptide and the targeting polypeptide is attached separately to the viral membrane, such a retroviral vector particle may be constructed by transfecting a packaging cell line such as those hereinabove described which includes polynucleotides encoding gag, pol, and env proteins, with expression plasmids including a first polynucleotide and a second polynucleotide. Examples of packaging cell lines include, but are not limited to, the PE501, PA317 (ATCC No. CRL 9078), ψ-AM, PA12, T19-14X, VT-19-17-H2, ψ CRE, ψ CRIP, GP+E—86, GP+envAM12, and DAN cell lines as described in Miller, Human Gene Therapy. Vol. 1, pgs. 5-14 (1990), and the ψ-2, C3A2, Q2bn, Q4dh, N-Pac, pHF-g, PM571, DSN, Omega E, Isolde, PG13 (U.S. Pat. No. 5,470,726), PG53, Haidee PhEB, Haidee PhEC, Haidee PhEE, Ampli GPE, BOSC23, GP7C-tTA-G10, FLYA13, FLYRD18, and FT67 cell lines as described in Coffin, et al., Retroviruses, Cold Spring Harbor Laboratory Press, pg. 449 (1997), which are incorporated by reference in their entirety. The first polynucleotide includes a nucleic acid sequence encoding the amphiphilic peptide hereinabove described and a nucleic acid sequence encoding at least a portion and, in one embodiment, all, of the membrane-spanning region of a transmembrane subunit of a viral envelope protein with or without nucleic acid sequences encoding the cytoplasmic tail. The second polynucleotide includes a nucleic acid sequence encoding a targeting polypeptide as hereinabove described and a nucleic acid sequence encoding at least a portion and, in one embodiment, all, of the membrane-spanning region of a transmembrane subunit of a viral envelope protein with or without nucleic acid sequences encoding a portion or all of the cytoplasmic tail region of the TM. A producer cell line then may be formed by means known to those skilled in the art. The resulting producer cells generate infectious retroviral vector particles that include the wild-type retroviral envelope protein and which exhibit altered receptor specificity and greater fusogenicity via the individually attached targeting peptide and amphiphilic fusion peptide, respectively. Alternatively, such viral particles including the attached targeting polypeptide may be generated by transfecting a prepackaging cell line with the first and second polynucleotides and a polynucleotide encoding wild-type envelope protein.

Alternatively, when a polypeptide including the amphiphilic peptide and the targeting peptide is attached to the viral membrane, the packaging cell line may be transfected with a single polynucleotide including a nucleic acid sequence encoding at least a portion and, in one embodiment, all, of a membrane-spanning region of a transmembrane subunit, a nucleic acid sequence encoding the amphiphilic peptide, a nucleic acid sequence encoding a spacer moiety, and a nucleic acid sequence encoding the targeting polypeptide. A producer cell then is formed by means known to those skilled in the art. The resulting producer cell generates viral particles that include a wild-type envelope protein, wherein the polypeptide including the amphiphilic peptide and the targeting polypeptide are attached separately to the viral membrane, whereby the amphiphilic peptide and the targeting polypeptide are exposed on the outside of the viral particle.

In another alternative, the retroviral vector particle may be constructed first by generating a wild-type retrovirus from a packaging cell line such as those hereinabove described. The amphiphilic peptide and the targeting polypeptide each are attached to the viral membrane, either by attachment to the membrane through a peptide-lipid conjugate as hereinabove described, or through a glycolipid linker. In yet another alternative, a polypeptide including the targeting polypeptide, the amphiphilic peptide, and a spacer moiety as hereinabove described, is attached to the viral membrane of a wild-type retroviral particle by means such as those hereinabove described.

In another embodiment of the present invention, there is provided a viral particle that does not include a naturally occurring or wild-type envelope protein or a modified envelope protein. In such an embodiment, there is provided an “artificial envelope protein” comprised of a targeting polypeptide and an amphiphilic peptide as hereinabove described. The targeting polypeptide and the amphiphilic peptide are attached to the viral membrane. Means of attachment include those hereinabove described. The targeting polypeptide and the amphiphilic peptide may be attached to the viral membrane as two independent peptides or as one polypeptide that provides both binding and fusion functions in tandem. The polypeptide also includes an appropriate spacer moiety placed between the targeting polypeptide and the amphiphilic peptide. Thus, the targeting polypeptide and the amphiphilic peptide are included as part of an “artificial envelope protein”. In another embodiment, one or more types of targeting and/or fusion promoting amphiphilic peptides may be included as part of the “artificial envelope protein” attached to the viral membrane for potentiating infection by the viral particle. Such may include the use of more than one amphiphilic peptide that promotes fusion and/or subsequent events in the infection of cells, resulting in the delivery of genetic material into the cell.

Such a viral particle, which includes an “artificial envelope protein,” may be generated by transfecting a pre-packaging cell line, including polynucleotides encoding the retroviral gag and pol proteins as hereinabove described, with a first polynucleotide including a nucleic acid sequence encoding the amphiphilic peptide and a nucleic acid sequence encoding a portion or all of the membrane-spanning region of the transmembrane subunit of a viral envelope protein with or without a nucleic acid sequence encoding the cytoplasmic tail region, and a second polynucleotide including a nucleic acid sequence encoding a targeting polypeptide and a nucleic acid sequence encoding a portion or all of the membrane-spanning region of the transmembrane subunit of a viral envelope protein with or without a nucleic acid sequence encoding the cytoplasmic tail region. Alternatively, the pre-packaging cell line is transfected with a single polynucleotide including a nucleic acid sequence encoding a portion or all of the membrane-spanning region of the transmembrane subunit, a nucleic acid sequence encoding the amphiphilic peptide, a nucleic acid sequence encoding a spacer moiety, and a nucleic acid sequence encoding the targeting polypeptide. A producer cell line then may be formed by transfecting the pre-packaging cell line with an appropriate retroviral expression plasmid, such as those herein described.

Upon transfection of the pre-packaging cell with the appropriate polynucleotide(s),and an appropriate retroviral expression plasmid as described herein to form a producer cell, the producer cell generates viral particles which include an “artificial envelope protein,” including the amphiphilic peptide and the targeting polypeptide, attached to the viral membrane. In one embodiment, each of the amphiphilic peptide and the targeting polypeptide is attached to the viral membrane separately. In another embodiment, a single polypeptide including the amphiphilic peptide and the targeting polypeptide is attached to the viral membrane.

Alternatively, a viral particle is generated from a prepackaging cell line. Such viral particle includes a viral membrane, but does not include a viral envelope protein. Upon generation of such viral particle, each of the amphiphilic peptide and the targeting polypeptide is attached to the viral membrane by means such as those hereinabove described, such as, for example, by attaching the amphiphilic peptide and the targeting polypeptide to the membrane through a peptide-lipid complex, or by attaching the amphiphilic peptide and the targeting polypeptide to a viral membrane via a glycolipid linker. Alternatively, a single polypeptide, including the amphiphilic peptide, a targeting polypeptide, and a spacer moiety, is attached to the viral membrane via chemical means such as those hereinabove described, to provide a viral vector particle having an “artificial envelope protein” including the amphiphilic peptide and the targeting polypeptide.

Because the “artificial envelope protein” has a significantly reduced amount of material that is derived from a retroviral envelope protein, a viral particle having such an “artificial envelope protein” is less likely to elicit an immune response than a viral particle that retains all or a majority of the wild-type envelope protein structure.

Thus, the amphiphilic peptides of the present invention are employed in the formation of a variety of viral vectors or viral particles having modified viral envelopes or “artificial envelope proteins.” The use of such vectors employing amphiphilic peptides, derivatives or analogues of the present invention for increasing the expression of a heterologous gene transfected into a cell with the help of such a vector is contemplated by the present invention. Preferred vectors contemplated by the present invention are such vectors that increase the expression of a heterologous gene by more than 10fold, as compared to a suitable control, such as a corresponding vector that does not employ an amphiphilic peptide, derivative or analogue of the present invention.

The “artificial envelope” of the viral particle can be generated via expression of the targeting and fusion peptides on the surface of the viral particle as hereinabove described. Alternatively, an artificial surface may be generated, for example, as an artificial bilayer used to envelop viral particles derived by any means. This constitutes the generation of artificial virusomes that can be retargeted and/or engineered to have enhanced fusion or other entry parameters due to the new encapsulating surface. In such embodiments, the amphiphilic peptides described herein or analogues thereof may serve a variety of functions. For example, the peptide may function as a fusion potentiating molecule. In addition, the peptides provide for more efficient incorporation of external polypeptides into a viral surface coat. This is achieved by attaching an external polypeptide to a transmembrane protein or peptide, and attaching the amphiphilic peptide on the cytoplasmic side, whereby the amphiphilic peptide provides for structurally favorable association with core proteins of the virion, thereby potentiating favorable surface expression of the external peptide.

The targeting polypeptide, which may be included in the various embodiments of the vector particles hereinabove described, includes a binding region that binds to a receptor located on a desired cell type. Such targeting polypeptides include, but are not limited to, antibodies and fragments thereof, including single-chain antibodies, monoclonal antibodies, and polyclonal antibodies. Such antibodies include, but are not limited to, antibodies and fragments or portions thereof which bind to erb-B2, such as, for example, e23 antibody; antibodies which bind to receptors such as, for example, the CD4 receptor on T-cells; antibodies which bind to the transferring receptor; antibodies directed against human leukocyte antigen (HLA); antibodies to carcinoembryonic antigen; antibodies to placental alkaline phosphates found on testicular and ovarian cancer cells; antibodies to high molecular weight melanoma-associated antigen; antibodies to polymorphic epithelial mucin found on ovarian cancer cells; antibodies to human chronic gonadotropin; antibodies to CD20 antigen of B-lymphoma cells; antibodies to alphafetoprotein; antibodies to prostate specific antigen; OKT-3 antibody, which binds to CD3 T-lymphocyte surface antigen; antibodies which bind to B-lymphocyte surface antigen; antibodies which bind to EGFR (c-erb-B1 or c-erb-B2) found on glioma cells, B-cell lymphoma cells, and breast cancer cells; anti-tac monoclonal antibody, which binds to the Interleukin-2 receptor; anti-transferrin monoclonal antibodies; monoclonal antibodies to gp 95/gp 97 found on melanoma cells; monoclonal antibodies to p-glycoproteins; monoclonal antibodies to cluster-1 antigen (N-CAM), cluster-w4, cluster-5A, or cluster-6 (LeY), all found on small cell lung carcinomas; monoclonal antibodies to placental alkaline phosphates; monoclonal antibodies to CA-125 found on lung and ovarian carcinoma cells, monoclonal antibodies to epithelial specific antigen (ESA) found on lung and ovarian carcinoma cells; monoclonal antibodies to CD19, CD22, and CD37 found on B-cell lymphoma cells; monoclonal antibodies to the 250 kDa proteoglycan found on melanoma cells; monoclonal antibodies to p55 protein found on breast cancer cells; monoclonal antibodies to the TCR-IgH fusion protein found on childhood T-cell leukemia cells; antibodies to T-cell antigen receptors; antibodies to tumor specific antigen on B-cell lymphomas; antibodies to organ cell surface markers; anti-HIV antibodies, such as anti-HIV gp 120-specific immunoglobulin, and anti-erythrocyte antibodies.

Other targeting peptides which may be employed include cytokines. Such cytokines include, but are not limited to, interleukins, including Interleukin-1α, Interleukin-1β, and Interleukins 2 through 14; growth factors such as epithelial growth factor (EGF), TGF-α, TGF-β, fibroblast growth factor (FGF), keratinocyte growth factor (KGF), PDGF-A, PDGF-B, PD-ECGF, IGF-I, IGF-II, and nerve growth factor (NGF), which binds to the NGF receptor of neural cells; colony stimulating factors such as GM-CSF, G-CSF, and M-CSF, leukemia inhibitory factor (LIF); interferon's such as interferon-α, interferon-β, and interferon-γ; inhibin A; inhibin B; chemotactic factors; α-type intercrine cytokines; and β-type intercrine cytokines.

Still other targeting polypeptides which may be employed include, but are not limited to, melanoma stimulating hormone (MSH), which binds to the MSH receptor on melanoma cells; peptidomimetic analogues of α-MSH, including a peptidomimetic analogue having the structure Ser-Tyr-Ser-Nle-Glu-His-(D-Phe)-Arg-Trp-Gly-Lys-Pro-Val, wherein Nle is norleucine and D-Phe is a D-phenylalanine residue; the polypeptide FLA16, which has the sequence Cys-Gln-Ala-Gly-Thr-Phe-Ala-Leu-Arg-Gly-Asp-Asn-Pro-Gln-Gly-Cys, which binds to the integrins VLA3, VLA4, and VLA5 found on human histiocytic lymphoma cells; the polypeptide having the structure Gly-Glu-Arg-Gly-Asp-Gly-Ser-Phe-Phe-Ala-Phe-Arg-Ser-Pro-Phe, which binds to the integrin αvβ, found on melanoma cells; erythropoietin, which binds to the erythropoietin receptor; adhering; selections; CD34, which binds to the C34 receptor of hematopoietic stem cells; CD33, which binds to premyeloblastic leukemia cells; stem cell factor; asialoglycoproteins, including asialoorosomucoid, asialofetuin, and alpha-1 acid glycoprotein, which binds to the asialoglycoprotein receptor of liver cells; insulin; glucagon; gastric polypeptides, which bind to receptors on hematopoietic stem cells; C-kit ligand; tumor necrosis factors (or TNF's) such as, for example, TNF-alpha and TNF-beta; ApoB, which binds to the LDL receptor of liver cells; alpha-2-macroglobulin, which binds to the LRP receptor of liver cells; mannose-containing peptides, which bind to the mannose receptor of macrophages; sialyl-Lewis-X antigen-containing peptides, which bind to the ELAM-1 receptor of activated endothelial cells; CD40 ligand, which binds to the CD40 receptor of B-lymphocytes; ICAM-1, which binds to the LFA-1 (CD11b/CD18) receptor of lymphocytes, or to the Mac-1 (CD11a/CD18) receptor of macrophages; M-CSF, which binds to the c-fms receptor of spleen and bone marrow macrophages; VLA-4, which binds to the VCAM-1 receptor of activated endothelial cells; LFA-1, which binds to the ICAM-1 receptor of activated endothelial cells; HIV gp120 and Class II MHC antigen, which bind to the CD4 receptor of T-helper cells; foliates and somatostatin, which bind to foliate and somatostatin receptors, respectively, of liver cells; and the LDL receptor binding region of the apolipoprotein E (ApoE) molecule. It is to be understood, however, that the scope of the present invention is not to be limited to any specific targeting polypeptide.

In one embodiment, the targeting polypeptide is a single chain antibody.

In another embodiment, the targeting polypeptide includes a binding region that binds to an extracellular matrix component. The term “extracellular matrix component,” as used herein, means a molecule that occupies the extracellular spaces of tissues. Such extracellular matrix components include, but are not limited to, collagen (including collagen Type I and collagen Type IV), laminin, fibronectin, elastin, glycosaminoglycans, proteoglycans, and sequences which bind to fibronectin, such as arginine-glycine-aspartic acid, or RGD, sequences. Binding regions that bind to an extracellular matrix component, and which may be included in a targeting polypeptide, include, but are not limited to, polypeptide domains that are functional domains within von Willebrand Factor or derivatives thereof, wherein such polypeptide domains bind to collagen. In one embodiment, the binding region is a polypeptide having the following structural formula: Trp-Arg-Glu-Pro-Ser-Phe-Met-Ala-Leu-Ser.

Other binding regions that bind to an extracellular matrix component, and which may be included in the viral envelope, include, but are not limited to, the arginine-glycine-aspartic acid, or RGD, sequences, which binds fibronectin, and a polypeptide having the sequence Gly-(Gly-Trp-Ser-His-Trp, which also binds to fibronectin.

It is to be understood, however, that the scope of the present invention is not to be limited to any specific targeting polypeptide, binding region, or ligand to which the targeting polypeptide may bind.

In a preferred embodiment, the viral vector or viral particle further includes at least one polynucleotide encoding a heterologous polypeptide that is to be expressed in a desired cell. The heterologous polypeptide may, in one embodiment, be a therapeutic agent. The term “therapeutic” is used in a generic sense and includes treating agents, prophylactic agents, and replacement agents.

It is to be understood, however, that the scope of the present invention is not to be limited to any particular therapeutic agent.

Accordingly, the uses of the peptides or derivatives and analogues of the invention, or a nucleic acid encoding such a peptide, for the preparation of a medicament, are within the scope of the present invention.

The polynucleotide encoding the therapeutic agent is under the control of a suitable promoter. Suitable promoters that may be employed include those known to those skilled in the art, including, but are not limited to, the retroviral LTR; the SV40 promoter; the cytomegalovirus (CMV) promoter; and the Rous Sarcoma Virus (RSV) promoter. The promoter also may be the native promoter that controls the polynucleotide encoding the therapeutic agent. It is to be understood, however, that the scope of the present invention is not to be limited to specific foreign genes or promoters.

In a preferred embodiment, the polynucleotide encoding a therapeutic agent may be contained in a retroviral expression plasmid, which is transfected into the appropriate packaging or prepackaging cells hereinabove described, to form producer cells that generate the vector particles hereinabove described.

In one embodiment, the retroviral expression plasmid may be derived from Moloney Murine Leukemia Virus and is of the LN series of vectors, such as those hereinabove mentioned, and described further in Bender, et al., J. Virol., Vol. 61, pgs. 1639-1649 (1987) and Miller, et al., Biotechniques, Vol. 7, pgs 980-990 (1989).

In another embodiment, the retroviral expression plasmid may include at least four cloning, or restriction enzyme recognition sites, wherein at least two of the sites have an average frequency of appearance in eukaryotic genes of less than once in 10,000 base pairs; i.e., the restriction product has an average DNA size of at least 10,000 base pairs. Such plasmids are further described in U.S. Pat. No. 5,672,710 incorporated herein by reference in its entirety.

The retroviral expression plasmid includes one or more promoters for the genes contained in the vector. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and β-actin promoters). Other viral promoters that may be employed include, but are not limited to, adenovirus promoters, TK promoters, and B19 parvovirus promoters. The selection of a suitable promoter will be apparent to those skilled in the art from the teachings contained herein.

The viral vectors or viral particles, which include the amphiphilic peptide hereinabove described and may further include a targeting polypeptide, and a polynucleotide encoding a therapeutic agent, may be administered to a host in an amount effective to produce a therapeutic or beneficial effect in the host. The term “beneficial effect,” as used herein, means that the effect is less than curative, but improves the quality of life in the host, such as, for example, alleviating a medical condition. The host may be a mammalian host, which may be a human or non-human primate host. The viral vectors or viral particles, upon administration to the host, travel to and transduce the desired cells, whereby the transduced target cells express the therapeutic agent in vivo. The exact dosage of viral vectors or viral particles that may be administered is dependent upon a variety of factors, including the age, sex, and weight of the patient, the target cells which are to be transduced, the therapeutic agent that is to be administered, and the severity of the disease or disorder to be treated.

Compositions suitable for medical treatment that include a peptide of the invention or a viral or synthetic vector of the invention, are also within the scope of the present invention.

The viral vectors or viral particles or compositions including such viral vectors or viral particles may be administered to the host systemically, such as, for example, by intravenous, intraperitoneal, intracolonic, intratracheal, endotracheal, intranasal, intravascular, intrathecal, intraarterial, intracranial, intramarrow, intravesicular, intrapleural, intradermal, subcutaneous, intramuscular, intraocular, intraosseous, and intrasynovial administration. The viral vectors or viral particles also may be administrated topically.

Cells that may be transduced with the viral vectors or viral particles of the present invention include, but are not limited to, primary cells, such as primary nucleated blood cells, primary tumor cells, endothelial cells, epithelial cells, vascular cells, keratinocytes, stem cells, hepatocytes, chondrocytes, connective tissue cells, fibroblasts and fibroelastic cells of connective tissues, mesenchymal cells, mesothelial cells, and parenchymal cells; smooth muscle cells of the vasculature; hematopoietic stem cells; T-lymphocytes; B-lymphocytes; neutrophils; macrophages; platelets; erythrocytes; reparative mononuclear granulocytic infiltrates of inflamed tissues; nerve cells; brain cells; muscle cells; osteocytes and osteoblasts in bone; lung cells, pancreatic cells; epithelial and subepithelial cells of the gastrointestinal and respiratory tracts; and malignant and non-malignant tumor cells. The selection of the particular cells which are to be transduced is dependent upon the disease or disorder to be treated as well as the targeting polypeptide. Such cells may be transduced in vivo, or may be transduced ex vivo, and then administered to a host in an amount effective to provide a therapeutic effect or a beneficial effect. It is to be understood that the scope of the present invention is not to be limited to the transduction of any specific cells.

When the viral vectors or viral particles include a targeting polypeptide that binds to an extracellular matrix component, such viral vectors or viral particles may be employed in treating diseases or disorders associated with an exposed extracellular matrix component. Such diseases or disorders include, but are not limited to, cardiovascular diseases; cirrhosis of the liver; connective tissue disorders (including those associated with ligaments, tendons, and cartilage); and vascular disorders associated with the exposition of collagen. The vector particles may be used to deliver therapeutic genes to restore endothelial cell function and to combat thrombosis, in addition to limiting the proliferative and fibrotic responses associated with neointima formation. The vector particles also may be employed in treating vascular lesions; ulcerative lesions; areas of inflammation; sites of laser injury, such as the eye, for example; sites of surgery; arthritic joints; scars; and keloids. The viral vectors or viral particles also may be employed in wound healing.

In addition, viral vectors or viral particles which include a targeting polypeptide that binds to an extracellular matrix component also may be employed in the treatment of tumors, including malignant and non-malignant tumors. Although Applicants do not intend to be limited to any theoretical reasoning, tumors, when invading normal tissues or organs, secrete enzymes such as collagenases or metalloproteinases that expose extracellular matrix components. By targeting viral vectors or viral particles to such exposed extracellular matrix components, the vectors or particles become concentrated at the exposed matrix components that are adjacent the tumor, whereby the vector particles then infect the tumor cells. Such tumors include, but are not limited to, carcinomas; sarcomas, including chondrosarcoma, osteosarcoma, and fibrosarcoma; and brain tumors. For example, a viral vector or viral particle, including the amphiphilic peptide including a targeting polypeptide that binds to an extracellular matrix component located at a tumor site, and a polynucleotide encoding a negative selective marker or “suicide” gene, such as, for example, the Herpes Simplex Virus thymidine kinase (TK) gene, may be administered to a patient, whereby the viral vector transduces the tumor cells. After the tumor cells are transduced with the vector, an interaction agent or prodrug, such as gancyclovir or acyclovir, is administered to the patient, whereby the transduced tumor cells are killed.

It is to be understood that the present invention is not to be limited to the treatment of any particular disease or disorder.

The viral vectors or viral particles, which include the amphiphilic peptide, and may further include a targeting polypeptide, and a polynucleotide encoding a therapeutic agent, may be administered to an animal in vivo as part of an animal model for the study of the effectiveness of a gene therapy treatment. The vectors or particles may be administered in varying doses to different animals of the same species, whereby the vector particles will transduce the desired target cells in the animal. The animals then are evaluated for the expression of the desired therapeutic agent in vivo in the animal. From the data obtained from such evaluations, one may determine the amount of vector particles to be administered to a human patient.

The viral vectors or viral particles of the present invention also may be employed in the in vitro transduction of desired target cells, which are contained in a cell culture containing a mixture of cells. Upon transduction of the target cells in vitro, the target cells produce the therapeutic agent or protein in vitro. The therapeutic agent or protein then may be obtained from the cell culture by means known to those skilled in the art.

The viral vectors also may be employed for the transduction of cells in vitro in order to study the mechanism of the genetic engineering of cells in vitro.

In another embodiment, the amphiphilic peptide, and the targeting polypeptide if desired, is incorporated into or attached to the surface of a drug delivery or nucleic acid delivery vehicle (e.g., a nanoparticle) or incorporated into or attached to the surface of an encapsulating vesicle such as a liposome. In such an embodiment, the peptide forms a portion of the particle or of the encapsulating vesicle. The peptide may be bound to the particle covalently or non-covalently, and such bonding may be achieved by physical or chemical means, including but not limited to those hereinabove described.

In one embodiment, the amphiphilic peptides may be associated with a liposome bilayer. The peptides may be attached or incorporated into the inner and/or outer surfaces of the liposome bilayer by means known to those skilled in the art, such as by covalent bonding, or by linker moieties or by other means. The attachment of the peptide!s to the liposome may be to the phospholipids, lipids, lipid intricolating molecules, lipid modification molecules, or by any other means which allows surface association. The liposomes that include the peptides or analogues thereof may be employed for the enhanced delivery of therapeutic agents or polynucleotides to cells, or to interstitial spaces and other locations. The peptides or analogues thereof aid in fusing the liposome to desired cells or in releasing encapsulated therapeutic agents at a desired site.

In another embodiment, the amphiphilic peptides may be associated with polycations or cationic polymers, such as e.g. protamine, polyethylimine or polylysine. Polycations or cationic polymers are useful for condensing nucleic acids. Accordingly, in a further embodiment of this invention, the amphiphilic peptides may be associated with cationic lipid complexes of nucleic acids.

Polynucleotides encoding therapeutic agents, which may be contained in the liposome or the cationic lipid complex, include, but are not limited to, those described herein.

In general, the use of peptides of the invention is contemplated in combination with either viral vectors or synthetic vectors, as well as with hybrid synthetic and viral vectors, such as viral vectors that are chemically modified after they have been produced by a suitable producer cell.

The amphiphilic peptides of the present invention also may be employed as antibiotics, or anti-viral agents, or antimicrobial agents, whereby such peptides reduce, inhibit, prevent, or destroy the growth of a cell, virus, or virally-infected cell.

The peptides may be administered in vivo or in vitro. The peptides also may be administered directly to a target cell, virus, or infected cell, or the peptides may be administered systemically, directly or as conjugated to delivery vehicles. The polyvalent presentation on a surface of particles presenting the peptides is likely to potentiate the therapeutic or beneficial effect of the amphiphilic peptide or the analogues.

The peptides of the present invention allow a method for treating or controlling microbial infection caused by organisms that are sensitive to the peptides. Such treatment may comprise administering to a host organism or tissue susceptible to or affiliated with a microbial infection an antimicrobial amount of at least one of the peptides.

Because of the antibiotic, antimicrobial, and antiviral properties of the peptides, they may also be used as preservatives or sterilants of materials susceptible to microbial or viral contamination.

The pepticle(s) of the present invention may be administered to a host; in particular a human or non-human animal, in an effective antibiotic and/or anti-tumor and/or anti-viral and/or anti-microbial and/or anti-fungal and/or anti-parasitic amount.

### EXAMPLES

The invention now will be described with respect to the following examples; however, the scope of the present invention is not intended to be limited thereby.

### Example 1

### Materials and Methods

Cell lines

NIH3T3, 293T, and XC cells were obtained from the ATCC repository. XC6 cells are a hyperfusogenic line subcloned from XC cells. The 293/12 cell line is a 293 cell sub-line expressing ecotropic receptor protein ATRC-1 (also known as MCAT1) (Ragheb et al., J. Virol., Vol. 69, pgs. 7205-7215, (1995)). Cells were maintained in D10: Dulbecco's modified essential medium, (Cell Culture Core Facility, USC), 10% fetal calf serum (FCS), 2 mM glutamine.

Peptides

Melittin was obtained from Sigma Chemical Co. (St. Louis, Mo.). Peptides were synthesized and HPLC-purified at the USC Norris Core Facility. Sequences were verified by mass spectroscopy. Peptide names indicate the first and the last residue number corresponding to the Moloney Murine Leukemia Virus (MoMuLV) env amino acid sequence. If the peptide has a residue different from the wild type MoMuLV env sequence, the wild type residue is listed followed by the number of the residue followed by the mutated amino acid (eg., 598-616 R609C). Aliquots of the stock solution for single use (2 mg/ml in ddH ₂O or in 50% dimethylsulfoxide) were stored at −20° C. Peptides containing single cysteine residues were modified with the sulfhydryl-selective reagent (1-oxyl-2,2,5,5 tetramethyl-pyroline-3-methyl)-methane thiosulfonate (Reanal, Budapest, Hungary) to generate a spin-labeled side chain referred to as R1. The reaction was carried out as described (Mchaourab et al., Biochemistry, Vol. 35, pgs. 7692-7704 (1996)), and the peptides purified by reverse-phase HPLC.

Liposome Preparation

Liposomes were prepared from 1-Palmitoyl-2-Oleoyl-sn-Glycero-3-(Phospho-rac-(1-glycerol)) (POPG) and 1-Palmitoyl-2-Oleoyl-sn-Glycero-3-Phosphocholine (POPC) (Avanti Polar Lipids Inc., Birmingham, Ala.). The lipid-chloroform solutions were mixed at a molar ratio of 1:3 (POPG:POPC), dried under N₂ and vacuum, sonicated, and taken through freeze-thaw cycles repeated a minimum of 5 times.

Circular Dichroism Analysis

The peptides for circular dichroism (CD) analysis were used at a final concentration of 50 μM. The stock solution of extruded liposomes at 50 mg/ml was used. The final peptide-to-lipid molar ratio was 1/100 in 20 mM Na₂PO₄ buffer. CD spectra were obtained using a Jasco J-600 CD spectrometer. Samples were scanned from 250 nm to 195 nm using cuvettes with a path length of 1 mm. Each result represents an average of 9 scans.

Electron Parymagnetic Resonance Analysis Of Spin Labeled Peptides

Spin labeled peptides (50 μM) diluted in buffer (50mM NaPO₄, 100 mM NaCl, pH 7.4) were added to the extruded liposomes at a peptide-to-lipid molar ratio of greater than 1/100. X-band electron paramagnetic resonance (EPR) spectra were recorded at room temperature. The spin accessibility parameter (II) was determined for 20 mM nickel ethylenediaminediacetic acid, or NiEDDA, and O₂ in equilibrium with air as previously described (Farahbakhsh, et al., Photochemistry and Photobiolocy, Vol. 56, pgs. 1019-1033 (1992)). The topology or context parameter φ was calculated as φ=ln [II(O₂)/II(NiEDDA)] (Altenbach, et al., Proc. Nat. Acad. Sci., Vol. 91, pgs. 1667-1671 (1994)).

Electrophysiological Procedures

Peptide-induced K′ release from liposomes. Liposomes (see above) were made in 100 nM KCl, 10 mM Tris-HCl, pH 7.5. The external KCl was reduced to less than 0.1 mM by two passages through a PD10 column (Pharmacia Biotech AB, Uppsala, Sweden) equilibrated in NaCl buffer (100 mM NaCl, 10 mM Tris-HCl, pH 7.5). Potassium release from liposomes was detected by a K′-selective microelectrode (Micro-electrode Inc.) and recorded on VHS tape using a pulse-code modulator. Peptides were assayed at 10 μM and 30 μM. For the planar lipid bilayer integrity assay done as in Lin, et al., J. Biol. Chem., Vol. 272, pgs. 44-47 (1997), 500 μm diameter black membrane was formed from a 15 mg/ml solution of POPG/POPC (1/3) in n-heptane as described in Kagan et al., Meth. Enzymol., Vol. 235, pgs. 699-713 (1994). Membrane was kept at 50 mV in 10 mM KCl, 10 mM Tris-HCl, pH 7.5, and conductance was tested as a consequence of addition of 3 μM peptide.

Envelope Mutants

The envelope protein mutants were constructed in the ecotropic env expression vector pCEE⁺ (MacKrell, et al., J. Virol., Vol. 70, pgs. 1768-1774 (1996)) using the oligonucleotide-directed in vitro mutagenesis system Quickchange (Stratagene, La Jolla, Calif.). The name of the mutant reflects the amino acid position of the mutation with the wild type residue in one letter code on the left and the mutant residue on the right. An asterisk (*) represents a stop codon after the residue indicated. Construction of the mutants 616* (CEETR), 601*(CEET), 578* (CEET1), and GLA ecto (GLA 15E) was described (Ragheb, et al., J. Virol., Vol. 68, pgs. 3207-3219 (1994)). The previously published names are given here in parentheses; the new names are used for consistency in this example.

Chimeric env constructs containing either a melittin-like sequence, a hydrophilic sequence, or a random sequence were cloned into the CEE+ plasmid by overlap PCR oligonucleotide-directed in vitro mutagenesis using the version 2.1 Amersham kit (Arlington Heights, Ill.). The NotI/NsiI fragments from mutated plasmids were recloned into new CEE+ backbones and the cytoplasmic substitutions were verified by sequencing. The cytoplasmic env chimeras were introduced after I598. The melittin-like sequence is: LKVLTTGLPALM-SWIstop (the italicized M resulted from a PCR error of Ile to Met, because the sequence retained an amphiphilic character, it was used). The hydrophilic sequence is: HNRKLQHNKDRRSstop (the native hydrophobic amino acids were substituted by hydrophilic residues). The random sequence is: RFVNVNLRDYRFSDQSRLstop.

Transfection And Transduction Assays

NIH3T3 cells (1H10⁵) , 293T or 293/12 (1H10⁶) per 60 mm2 plates were transfected according to the Ca(PO₄)₂ transfection protocol of 5′-3′ Inc. (Boulder, Colo.) (day 1). For cell surface expression and co-culture experiments, 15 μg env expression plasmid was used. In transfections for transduction and Western Blot experiments 10 μg of env (MacKrell, et al., (1996)), 10 μg of pHIT 112 (gag-pol), and 10 μg of pHIT 60 (β-gal) (Soneoka, et al., Nucl. Acids Res., Vol. 23, pgs. 628-633 (1995)) expression plasmids were used. On day 2, D10 containing 1 mM sodium-butyrate was added for 8-12 hours, and replaced with 3 ml D10 for 24 hours. On day 3, the supernatant was passed through an 0.45 μm filter, serially-diluted, made to 8 μg/ml polybrene, and tittered for β-gal activity on NIH3T3 cells (as in Morgan, et al., J. Virol., Vol. 67, pgs. 4712-4721 (1993)).

Cell-To-Cell Fusion Assays For the end point fusion assay, 5×10⁵ XC6 cells were added to the env transfected 293 T cells 20 hrs. post-transfection, and stained with methylene blue (0.01% in methanol) at about 36 hours post-transfection for syncytia (cells with 4 or more nuclei) scoring. For the rate-of-fusion assay env plasmids were transfected into the ecotropic receptor-expressing 293/12 cells. The medium was changed on the day after transfection and scoring was done in live culture at 24, 26, 30, and 43 hours post-transfection. Results are represented as an average of syncytia number counted under the microscope on 10 separate 2 mm² grids.

Cell Surface Expression And The Analysis Of Viral Envelope Particle Incorporation Assays were done as described in Januszeski, et al., J. Virol., Vol. 71, pgs. 3613-3619 (1997).

### RESULTS

Structural Analysis Of The Peptide 598-616 Corresponding To The MoMuLV Env Cytoplasmic Tail Membrane Proximal Domain.

Computational Predictions Of The MoMuLV Env Cytoplasmic Membrane-Proximal Region Secondary Structure

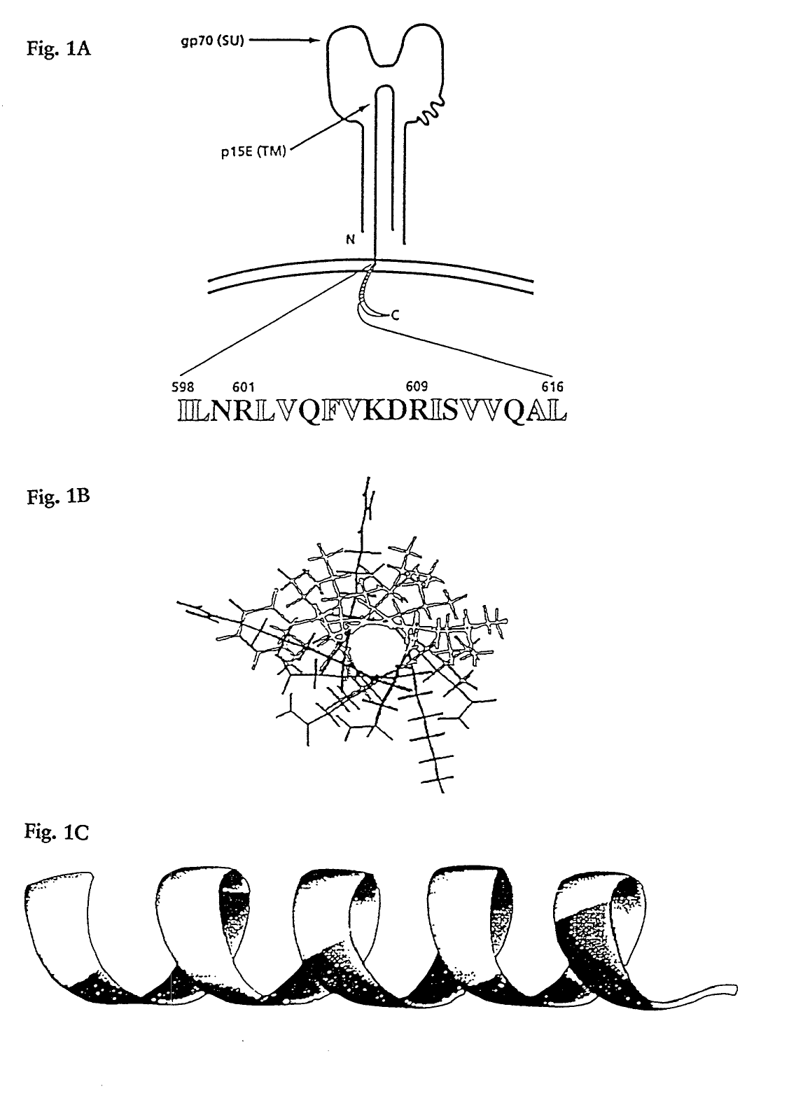

Although for a number of viral envelope ectodomains structural information has become available (Wilson, et al., Nature, Vol. 298, pgs. 366-373 (1981); Bullough, et al., Nature, Vol. 371, pgs. 37-43 (1994); Chan, et al., Cell, Vol. 89, pgs. 263-273 (1997); Fass, et al., Nature Structural Biology, Vol. 3, pgs. 465-469 (1996); Weissenhorn, et al., Nature, Vol. 387, pgs. 426-429 (1997)), structural information on env cytoplasmic domains has yet to be obtained. Thus, computational methods were applied initially to investigate the MoMuLV env cytoplasmic tail secondary structure. A schematic representation of the MoMuLV env is shown in FIG. 1A. A neural network program (Rost and Sander, Proteins, Vol. 19, pgs. 55-72(1994)) predicts that in a hydrophobic environment the membrane proximal region 598-616 of the env cytoplasmic tail will fold into an α—helix.

Algorithms which do not account for the polarity of the environment (Garnier, Kyte and Doolittle, Chou and Fasman) do not predict a helical structure. The helical-wheel method for visualizing amphiphilic_α-helixes (FIG. 1B) suggests a helical nature for the mature cytoplasmic tail. The method predicts a distinct amphiphilicity for the membrane-proximal segment 598-616, with residues positioned on either a hydrophobic or on a hydrophilic side of the helix in agreement with their polar characteristics, with the notable exception of Arg 609 (FIG. 1B, C). The predicted amphiphilic α helix 598-616 extends three residues into the presumed viral membrane-spanning region and is preceded by a predicted turn sequence of Gly-Pro-Cys. The amphiphilicity ends at residue 616, which also corresponds to the cleavage site of the R peptide.

Circular Dichroism (CD) Analysis Of Peptide 598-616 α-Helical Content

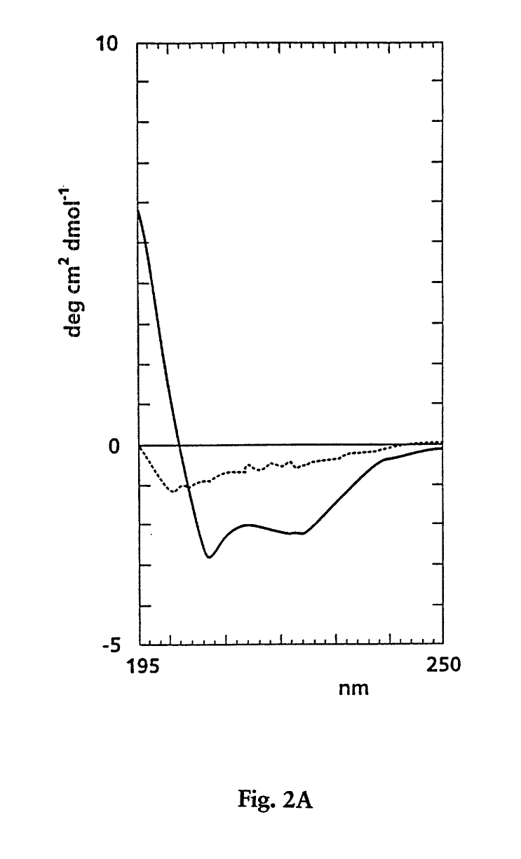

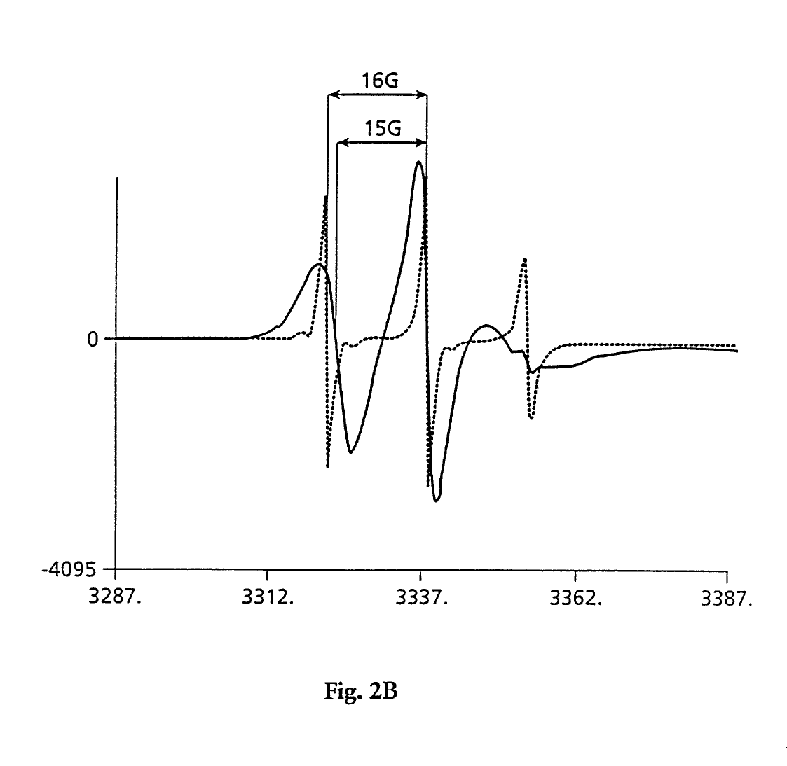

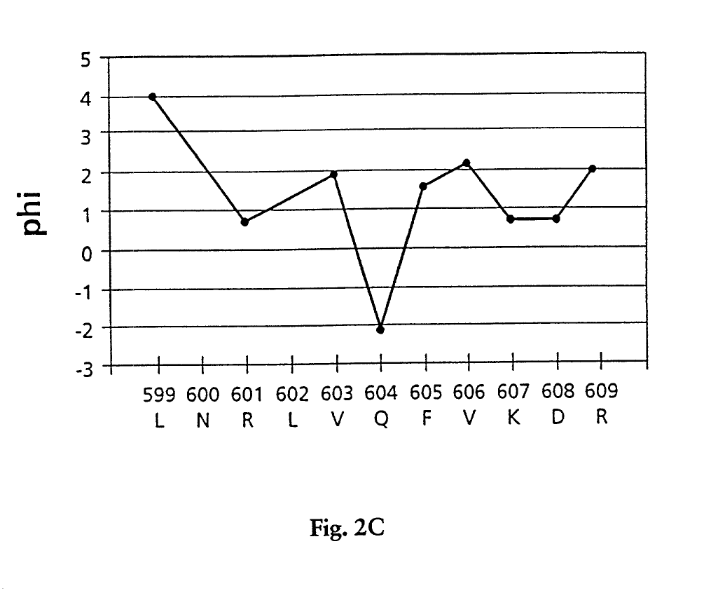

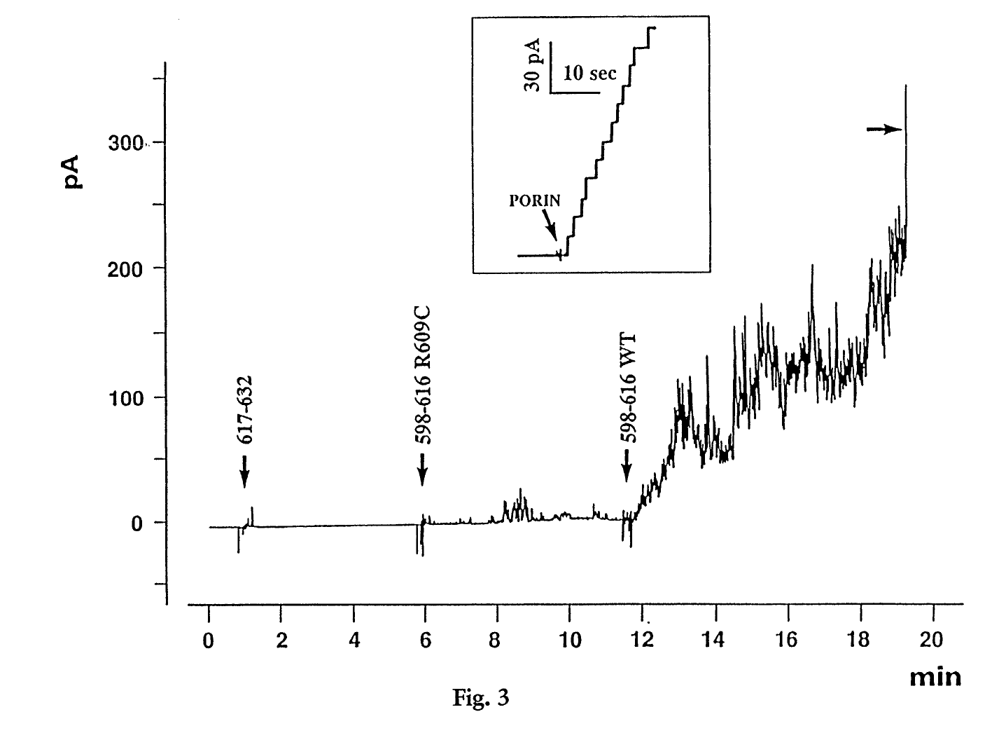

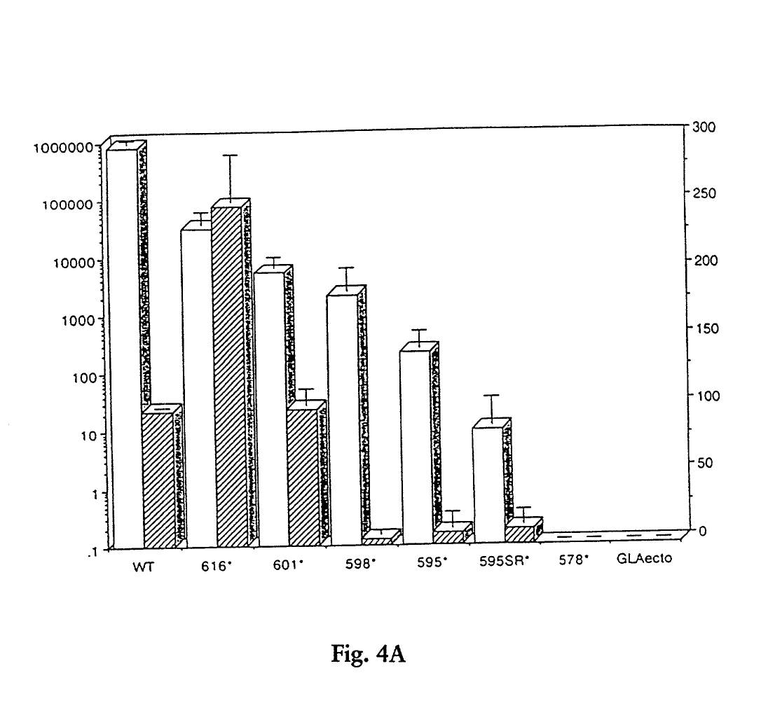

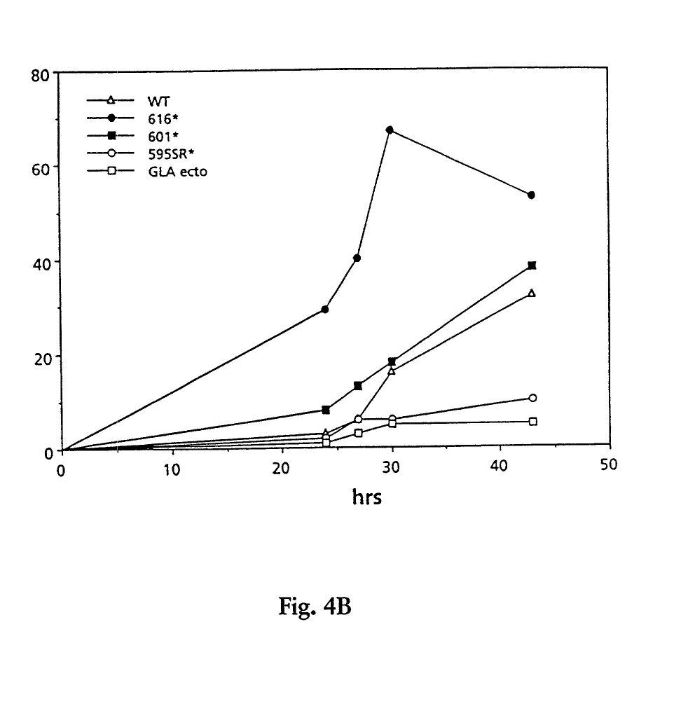

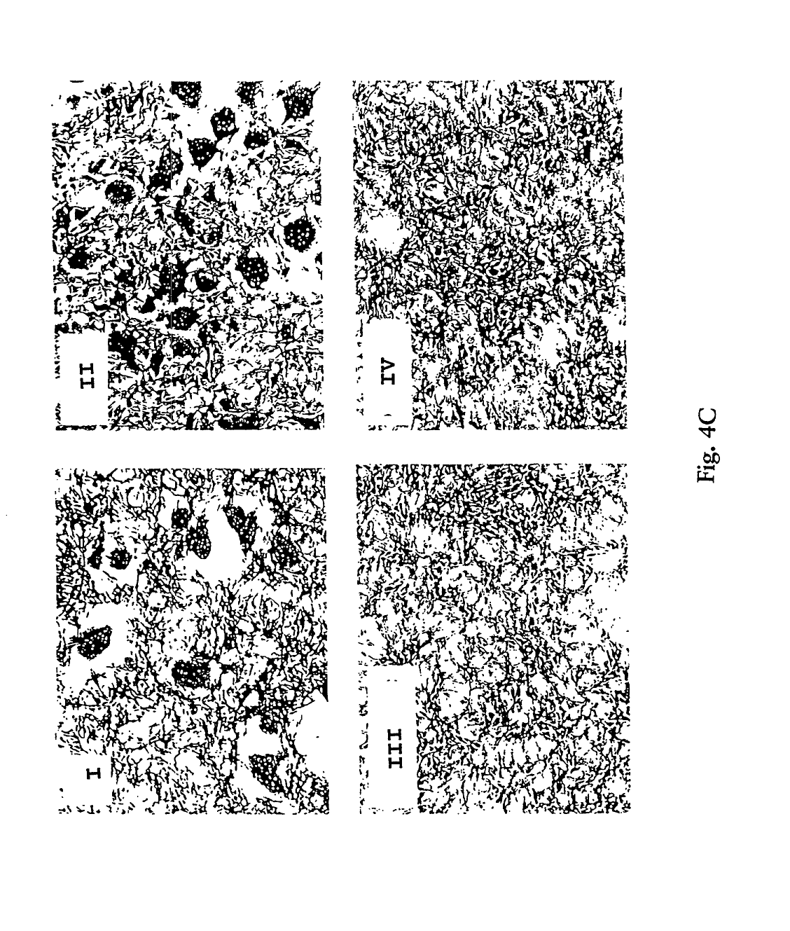

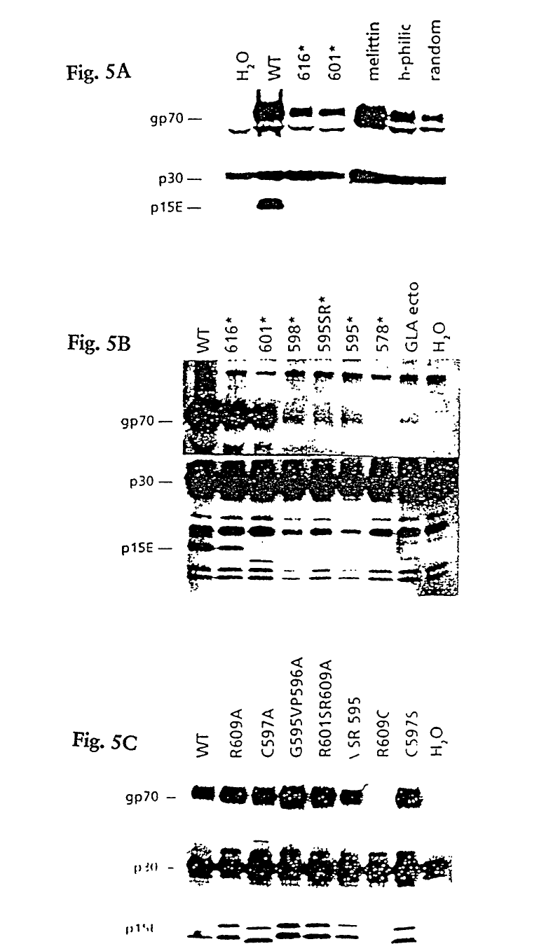

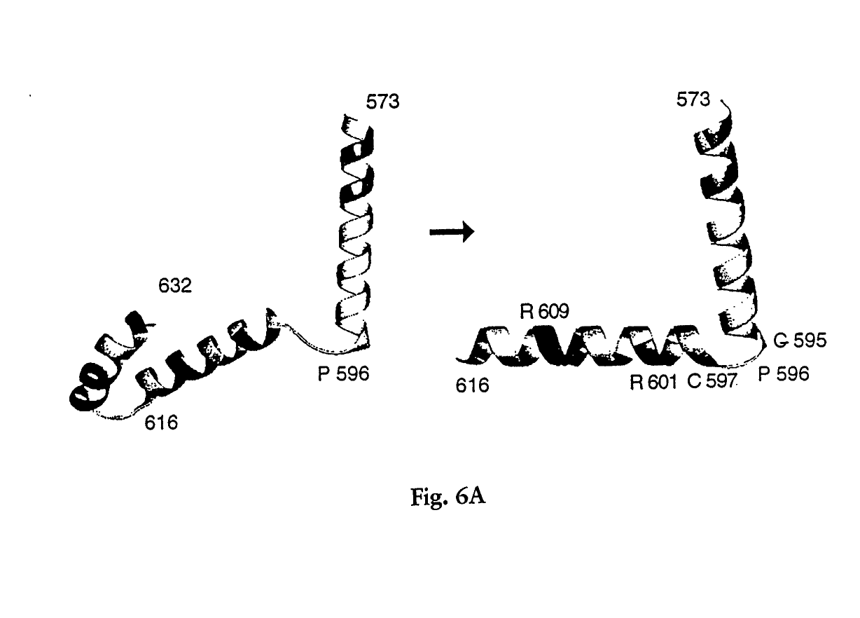

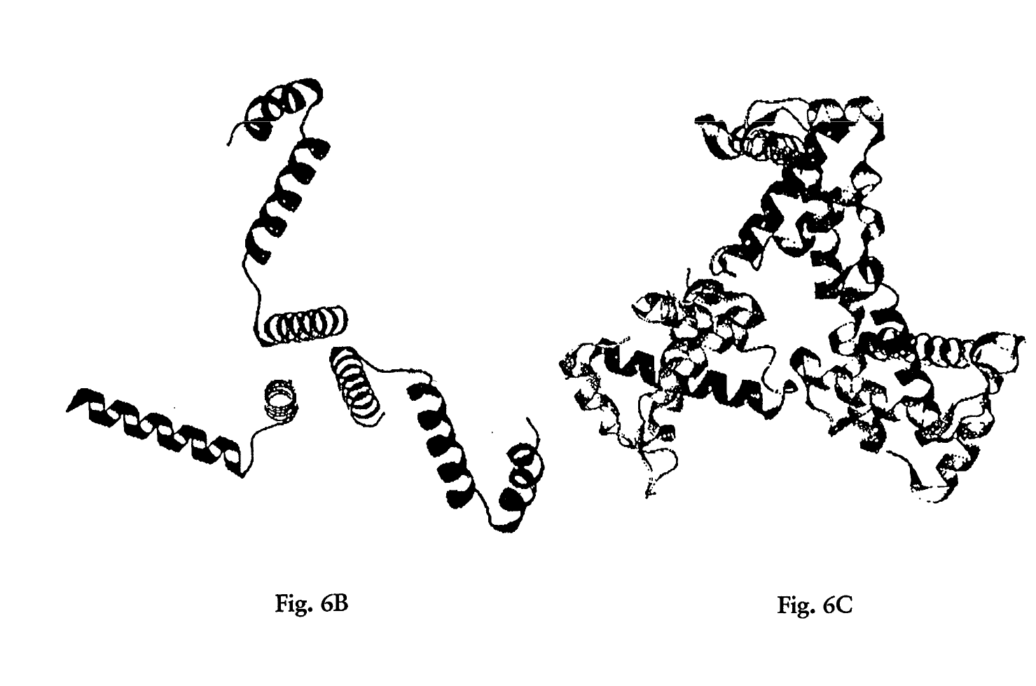

To analyze the secondary structure of the peptides corresponding to MoMuLV cytoplasmic tail segments, the peptides 598-616 and 617-632 were evaluated by CD spectroscopy. The CD spectrum produced by the peptide 598-616 absorption of polarized light in an aqueous environment is characteristic of a random coil conformation (FIG. 2A, dotted line). However, in the presence of a membrane environment provided by liposomes, peptide 598-616 becomes α-helical (FIG. 2A, solid line). The peptide 617-632 retained random coil conformation both in aqueous and in lipid environments (data not shown). The estimated percent α-helicity for peptide 598-616 is nearly 60%. Similar α-helical properties have been observed for the lytic amphiphilic segments from HIV-1 env cytoplasmic tail and the active component of bee venom, melittin (Eisenberg, et al., Biopolymers, Vol. 29, pgs. 171-177 (1992)). Thus, the computer-predicted α-helical structure for the region 598-616 is confirmed for peptide 598-616 by CD analysis in the presence of membranes.

Electron Paramagnetic Resonance Analysis Of Peptide 598-616

The structural features of the peptide 598-616 bound to membranes were investigated further by EPR analysis of corresponding peptides modified to contain single R1 nitroxide side chains at the positions indicated in FIG. 2C. For each labeled peptide in solution, the EPR spectrum consisted of three sharp resonance lines characteristic of a random coil for peptides containing R1 (Farahbakhsh, et al., Biochemistry, Vol. 34, pgs. 509-516 (1995)). A representative example is shown by the dotted line in FIG. 2B for the V606-R1 peptide. Upon addition to liposomes, the line shape is broadened considerably due to a reduction in motion of the R1 side chain, demonstrating interaction with the membrane (solid line, FIG. 2B). Judging from the line shapes, the contribution from the unbound peptide is very small, less than 1% (arrow FIG. 2B). All peptides 598-616 gave similar effects, although the individual line shapes varied (data not shown).

Spin-spin interactions between nitroxides result in spectral broadening over and above that due to motional effects (Mchaourab, et al., Biochemistry, Vol. 36, pgs. 307-316 (1997)). No evidence of such interaction was detected for any peptides 598-616 in membrane-bound state or solution, suggesting that, as tested, the peptides 598-616 do not aggregate but exist as monomers.

The topology and sequence-specific secondary structure of membrane-bound peptides and proteins can be determined from the accessibility of incorporated R1 side chains to collision with polar (NiEDDA) and non-polar (O₂) paramagnetic reagents in solution (Hubbell and Altenbach, Curr, Opin. in Struct. Biol., Vol 4, pgs. 566-573 (1994)). The accessibility is expressed by the quantity II, proportional to the collision frequency of the reagent with the nitroxide (Farahbakhsh, et al., Photochem. & Photobiol., Vol. 56, pgs. 1019-1033 (1992)). The topology parameter Φ=ln [II (O₂)/II(NieDDa)] is a quantitative measure of the depth of penetration of a nitroxide in a bilayer interior (Altenbach et al., Proc. Nat. Acad Sci., Vol. 91, pgs. 1667-1671 (1994)). A plot of Φ versus position of a single R1 side chain for the membrane-bound peptide is shown in FIG. 2C. Values of Φ>1 correspond to locations within the bilayer interior, values of Φ<0 correspond to locations in the aqueous phase, and values in the range of 0<Φ<1 correspond to interfacial locations. The data for the peptide 598-616 suggest that the spin-labeled residues 599,603, 605, 606, and 609 are within the bilayer interior, residues 601, 607, and 608 are interfacial, and residue 604 is clearly in the aqueous phase. As the position of the spin-labeled residue is sequentially advanced along the peptide 598-616, the oscillation in Φ values coincides roughly with the periodicity characteristic for an α-helix with 3.6 residues per turn (FIG. 2C). These assignments are consistent with an asymmetrically solvated amphiphilic α-helix.

Electrophysilogical Detection Of Peptide 598-616 Membrane Destabilizing Activity

Most of the lytic peptides such as defensins, magainin, alamethicin, melittin, etc., have amphiphilic character (Saberwal, et al., Biochemica et Biophysica Acta, Vol. 1197, pgs. 109-131 (1994)). The membrane activity of the peptide 598-616 was evaluated electrophysiologically by measuring membrane integrity in the presence of synthetic peptides. The results of such experiments are given in Table II below.

TABLE II

| Peptide Name^{a} | % K⁺-release^{b} |
|---|---|
| 0.1 M Triton X-100 | 100 |
| 617-632 | 0^{c} |
| 598-616 | 29.3 ± 3.6 |
| 598-616 R609C | 3.8 ± 1.5 |
| 598-616 R609A | 9.9 ± 1.8 |
| 598-616 R609 V V606R | 14 ± 2.4 |
| melittin | 30.9 ± 2.1 |

^{a}Peptide name reflects first and last residue corresponding to the position in MoMuLV env. The position of a mutation is shown with the wild type residue followed by the site of the mutation and the identity of the mutant residue.
^{b}Percentage of the intraliposomal K⁺ release induced by 10 mM peptides from liposomes (POPG/POPC 1:3, 10 mg/ml) loaded with 100 mM KC1 and dialyzed against 100 mM NaC1 buffer. The leakage of K⁺ was measured by a K⁺-sensitive electrode (wild type peptide 598-616, and mutant peptides 598-616 R609C and 598-616 V606R/R609V, R peptide 617-632 and melittin). Total K⁺ release was obtained with 0.1 M Triton X-100.
^{c}Activity that releases less than 2% K⁺ is not detectable.

^{a} Peptide name reflects first and last residue corresponding to the position in MOMuLV env. The position of a mutation is shown with the wild type residue followed by the site of the mutation and the identity of the mutant residue.

^{b} Percentage of the intraliposomal K⁺ release induced by 10 mm peptides from liposomes (POPG/POPC 1:3, 10 mg/ml) loaded with 100 mM KC1 and dialyzed against 100 mM NaCl buffer. The leakage of K⁺ was measured by a K⁺-sensitive electrode (wild type peptide 598-616, and mutant peptides 598-616 R609C and 598-616 V606R/R609V, R peptide 617-632 and melittin). Total K⁺ release was obtained with 0.1 M Triton X-100.

^{c} Activity that releases less than 2% K⁺ is not detectable.

A novel approach was developed to measure the membrane activity of peptide 598-616. Peptide-induced release of K⁺ from KCl-loaded lipid vesicles was detected using a K⁺-selective electrode. The addition of 10 μM wild type peptide 598-616 causes 29% K⁺ content release. At a concentration of 30 μM, about 70% K⁺ content is released from liposomes (data not shown). For comparison, 10 μM melittin, a lytic component of bee venom, causes 31% K⁺ content release. This result indicates that peptide 598-616 has a strong membrane destabilizing activity.

The EPR measurements and computer prediction suggest that Arg 609 in the segment 598-616 faces the membrane. To investigate the functional contribution of Arg 609, peptide 598-616 with mutations at position 609 were tested in membrane destabilization assays. The Arg 609 Cys mutation in peptide 598-616 lowered the level of K⁺ release by over 85% of the wild type peptide 598-616. Similarly, peptide 598-616 Arg 609 Ala lost 75% of its activity. The peptide 598-616 with the double mutation Val 606 Arg/Arg 609 Val was made to reposition Arg by one helical turn, but to retain the positive charge on the hydrophobic side of the amphiphilic helix. This peptide is membrane-active, although at about 50% activity relative to the wild-type.

Similarly strong membrane destabilizing activity was measured for peptide 598-616 when it was assayed for current induction across a voltage-clamped planar bilayer lipid membrane (FIG. 3). Peptide 598-616 causes increase in a non-selective planar membrane conductance that leads to membrane rupture. The substitution of Arg 609 by a Cys drastically reduces the peptide's membrane destabilizing activity (FIG. 3). The R-peptide (617-632) is inert in both the K⁺ release and the planar membrane assays. Together, the in vitro data suggest that an Arg positioned in the peptide 598-616 to face into the membrane contributes to membrane destabilization by this peptide.

In addition to substantiating the role of peptide 598-616 in membrane perturbation, the planar lipid membrane data suggest a molecular mechanism for membrane destabilization. If the peptide 598-616 were to form pores, an equal stepwise increase in planar membrane conductance that does not result in membrane rupture would be expected. Such is seen in the incorporation of uniform ion channels of Borrelia Burgdorferi porin protein (FIG. 3, insert) (Lin, et al., J. Biol. Chem., Vol. 272, pgs. 44-47 (1997)). The peptide 598-616, however, causes a chaotic membrane disruption process that culminates in membrane rupture (FIG. 3). This result is more consistent with a series of monomeric peptides associating with the membrane rather than with multimeric channel formation.

Mutagenic Analysis Of The MoMuLV Env Cytoplasmic Tail Membrane-Proximal Domain.

Progressive Truncations Into The Predicted Membrane-Proximal Helix Result In Progressive Loss Of Env Fusogenicity

Previous mutagenesis studies of MoMuLV env cytoplasmic tail demonstrated its contribution to fusion (Rein, et al., J. Virol., Vol. 68, pgs. 1773-1781 (1994); Ragheb, et al., J. Virol., Vol. 68, pgs. 3220-3231 (1994); Januszeski, et al., J. Virol., Vol. 68, pgs. 3613-3619 (1997); Thomas, et al., Virology, Vol. 227, pgs. 305-313 (1997)). To test the function of the region 598-616, a set of progressive truncations was tested. All of the truncated envelopes, except for 578*, are expressed efficiently in 293T cells. The fusogenicity of these mutants was assessed in two ways: (1) by an end-point syncytia formation (FIG. 4A, Appendix 1B), and (2) by analyzing rate of syncytia induction (FIG. 4B, Appendix 1B).

The end point fusion assay measures env-induced cell-to-cell fusion between env-transfected 293T cells and the ecotropic receptor expressing XC cells at 36 hrs post-transfection. The R-less (616*) envelope protein is the most fusogenic, over 2.5 times greater than the wild type envelope protein. The truncation at the presumed membrane spanning stop-transfer boundary at Arg 601 (601*) reduces envelope protein fusion activity to the level of the wild type envelope protein that retains the R peptide. The elimination of the remaining segment of the proposed α-helical structure in mutants 598*, 595*, and 595 Ser Arg* results in a dramatic decreace of fusion to near background levels.

The glycolipid-anchored envelope protein ectodomain (GLA ecto) and the tailless mutants except for the truncation retaining 8 residues of the membrane spanning region (578*) of the transmembrane subunit express on the cell surface (Appendix 1B). The glycolipid-anchored envelope protein ectodomain (GLA ecto) and the truncation retaining 8 residues of the membrane spanning region (578*) of the transmembrane subunit are fusion incompetent (Appendix 1B, FIG. 4A). The other envelope protein with cytoplasmic truncations exhibit reduced fusion compared to the R-less env. Thus, as measured by this end-point fusion co-culture assay, truncations retaining the full amphiphilic structure result in a maximum level of fusion, whereas the removal of the entire proposed membrane-proximal structure 598-616 decreases envelope protein-induced fusion drastically.

The rate of fusion is a more accurate method of assay fusogenicity of an envelope protein mutant because syncytia-to-syncytia fusion occurs in culture, thereby reducing the total number of syncytia present. The rate of fusion was determined by transfecting an env expression vector into the ecotropic receptor expressing 293/12 cells followed by periodic scoring of syncytia (FIG. 4B). The fusion kinetics of the 601* envelope protein are slightly faster than those of the wild type envelope protein, while rates of syncytia formation by the truncated envelope protein 595 Ser Arg* and the GLA ecto envelope protein are equal to the background. The syncytia formed by the 616* envelope protein at 24 to 30 hours post-transfection are 3 to 4 times more abundant than those formed by the wild type envelope protein. Thus, the rate of fusion indicates that removal of the region 598-616 affects envelope protein-mediated fusion adversely.

Progressive Truncation Of The Envelope Protein Cytoplasmic Tail Results In Progressive Loss Of Envelope Protein Incorporation And Transduction Efficiency

Because in some viruses the cytoplasmic tail region of the envelope protein has been speculated to interact with the matrix (Freed, et al., J. Virol., Vol. 69, pgs. 1984-1989 (1996); Vzorov, et al., Virology, Vol. 221, pgs. 22-33 (1996)), the effect of cytoplasmic truncations on the efficiency of incorporation of the envelope protein into viral particles and on titers was assessed. Virions were collected from the supernatant of 293T cells transfected with the env, β-gal , and the gag-pol expression plasmids, and analyzed for the level of envelope protein (SU gp 70 and TM p15E) by Western Blot (FIG. 5A, Appendix 1B).

The incorporation of the R-less envelope protein (616*) is considerably less efficient than that of the wild-type envelope protein (FIG. 5A, Appendix 1B), and is reduced slightly more in the case of the 601* envelope protein. The removal of the remaining residues of the proposed membrane-proximal functional structure in the envelope protein constructs 598*, 595*, 595 Ser Arg*, and GLA ecto, result in a dramatic reduction of envelope protein incorporation. The 578* envelope protein is not detected in virions.

Viral titer (FIG. 4A, white bars, Appendix 1B) is reduced 10 times for the 616* envelope protein virions, and decreased 100 times for the 601*envelope protein. The titers for 598*, 595*, and 595 Ser Arg* envelope protein-containing particles are reduced by three to four orders of magnitude. No titer was detected for the particles with 578* or GLA ecto envelope protein. Thus, the progressive truncations of the envelope protein cytoplasmic tail correlate with the progressive decrease of envelope protein incorporation and a subsequent progressive reduction in titer.

Mutations Of Arg 609.

The Electrophysiological data indicate that the efficient membrane-destabilizing ability of peptide 598-616 depends on the presence of Arg 609 (FIG. 3), and the EPR data suggest that Arg 609 faces the membrane (FIG. 2C). To examine the in vitro with the in vivo situation, two Arg 609 mutant env proteins, corresponding to the peptides assayed in vitro, were made and assayed (Appendix 1C). The Arg 609 Cys env mutant was not informative because it does not express efficiently. The efficiently expressed Arg 609 Ala env has one half the fusion activity, wild-type level of incorporation, and a normal titer. Similar data has been obtained with the C-terminal truncation mutants that extend past 609 in an earlier study (Januszeski, et al., 1997). These results suggest that in the context of the whole envelope Arg 609 potentiates, but is absolutely required for, fusion.

Mutations In Residues Gly 595, Pro 596, Cys 597 Decrease Env Fusion, Incorporation, And Transduction

Directly preceding the amphiphilic domain 598-616 are the residues Gly 595, Pro 596, Cys 597. Gly/Pro is a commonly observed turn sequence between two adjacent helixes (Efimov, et al., Molekuliarmaia Biologiya, Vol. 26, pgs. 1370-1376 (1992)). The cysteine in the analogous CAAX motif of other viruses is known to be lipid-modified. The potential structural contribution of Gly 595, Pro 596, and Cys 597 was assayed by conservative substitutions. In addition, the insertion of Ser and Arg before 595 (VSR595) was constructed to function as a membrane stop transfer signal. These mutants were expressed and incorporated into virions, but had slightly reduced fusion (Appendix 1C, FIG. 5B), indicating that none of these residues individually is essential for viral viability.

The MoMuLV Envelope Protein With A Substitution Of Region 598-616 By A Heterologous Amphiphilic α-Helix Retains Efficient Fusogenicity

The analysis of the in vitro data suggests that the envelope protein membrane-proximal region contributes to viral membrane destabilization due to its amphiphilic character. The results of cytoplasmic tail point mutagenesis, small deletions, and truncations done in this example and in that of Januszeski, et al. 1997, corroborate that the region 598-616 potentiates fusion, but do not determine directly whether the structure of the region is essential for its function in entry. To test the contribution of the env membrane-proximal region 598-616 to its function, this region was replaced with a heterologous amphiphilic α-helix. A sequence encoding a 15 residue segment from melittin, lacking its charged head, was used because melittin peptide was demonstrated to form an amphiphilic α-helix by X-ray crystallography, NMR, and EPR (reviewed in Dempsey, Biochimica et Biophysica Acta, Vol. 1031, pgs. 143-161 (1990)). This melittin fragment has near background activity in the K+ release Electrophysiological assay (data not shown). Thus, effects of substitutions with this sequence are expected to be due to its amphiphilic structure, and not to its lytic activity. For negative controls, the Moloney env 598-616 sequence was replaced by a random or by a hydrophobic sequence (See Materials and Methods).

To test highly fusogenic R-less env constructs, the env expression plasmids were transfected transiently into NIH 3T3 cells because of the low fusogenicity of these cells compared with the 293T/XC co-culture system. The data are normalized to the fusogenicity of 616*, the wild type R-less envelope protein (FIG. 4C, Appendix 1A). The hydrophilic and the random-tail chimeras form syncytia inefficiently, 1% and 8% compared to the R-less wild type (616*) fusion activity. The fusogenicity of these chimeras in the 293T/XC co-culture assays also is reduced severely compared to both R-less and the wild type envelope protein (data not shown). The Moloney/melittin chimeric envelope protein is at least as fusogenic as the 616* envelope protein. The removal of the cytoplasmic tail region up to the presumed membrane stop-transfer Arg 601 (601*) results in envelope protein with fusogenicity at least fourfold lower than that of the 616* envelope protein when measured in NIH 3T3 cells (Appendix 1A). All of the chimeric envelope protein constructs are expressed on the cell surface (Appendix 1A). Thus, the data indicate that potent fusion activity of R-less Moloney envelope protein (616*) is reduced when the hypothesized amphiphilic membrane-proximal region is shortened (601*), but retained if replaced by a heterologous segment from an amphiphilic peptide.

The efficiency of the envelope protein with cytoplasmic substitutions to mediate cell fusion was monitored by transient transfection of env constructs into NIH3T3 cells. Since in NIH3T3 cells there is no viral protease to cleave the R peptide, all of the chimeric env were engineered in the R-less form, to resemble the mature env cytoplasmic tail. All of the chimeric envelope protein constructs are expressed on the cell surface (Appendix 1C). Fusion of the R-less envelope protein was assayed by monitoring the formation of env-induced cell-to-cell fusion scored as syncytia in NIH3T3 due to the low fusogenicity of this cell line (FIG. 4C, Appendix 1C). The hydrophilic and the random-tail chimera form syncytia inefficiently (compare to the wild type 1% and 8% respectively), while the Moloney/melittin chimeric envelope protein is more fusogenic (139%) than the wild type R-less envelope protein (616*). The removal of the cytoplasmic tail up to the presumed membrane stop-transfer Arg 601 (601*) results in fusion competent envelope protein (Appendix 1C; Ragheb and Anderson, J. Virol., Vol. 68, pgs. 3220-3231 (1994)). However, the fusogenicity of envelope protein 601*is at least 4 fold lower than that of the envelope protein 616* or the Moloney/melittin chimeric envelope protein. Thus, the data indicate that potent fusion activity of R-less Moloney envelope protein (616*) is reduced when the hypothesized amphiphilic membrane-proximal region is shortened (601*), but retained if replaced by a heterologous segment from an amphiphilic peptide.

The MoMuLV Envelope Protein With A Substitution Of Region 598-616 With A Heterologous Amphiphilic α-Helix Efficiently Incorporates Envelope Protein Into Virions And Retains Wild Type Transduction Level

Virions containing envelope protein constructs with cytoplasmic substitutions were produced in 293T cells as described and were tested for efficiency of incorporation into viral particles (FIG. 5A, Appendix 1A). The Moloney/melittin envelope protein was incorporated efficiently into virions, while the incorporation of hydrophilic and random chimeric envelope protein was reduced. This result suggests that the secondary structure of the membrane-proximal region is important for incorporation.

The envelope protein constructs with cytoplasmic substitutions next were tested for their ability to transduce NIH3T3 host cells. Virions with the Moloney/melittin tail had near wild type transduction levels (3×10⁵ cfu/ml; Appendix 1A). The presence of the hydrophilic or the random tail reduced titer by two orders of magnitude. Thus, successful replacement of the region 598-616 by a heterologous amphiphilic α-helix indicates that the functional role of the envelope protein membrane-proximal domain is influenced by its secondary structure rather than by a specific sequence.

Discussion

Among the unresolved issues in the mechanism of viral entry is the question of how viruses induce an energetically highly unfavorable event of fusion between viral and host membranes. The results of this example indicate that the formation of a membrane-destabilizing amphiphilic α-helix 598-616 in the envelope protein cytoplasmic tail region potentiates envelope protein-mediated fusion. The release of the fusion peptide in the envelope protein ectodomain likely completes the membrane fusion. The data also suggest that the amphiphilic α-helix in the envelope protein cytoplasmic tail region contributes positively to the efficient incorporation of envelope protein into a viral particle.

A Conserved Amphiphilic Motif In Envelope Protein Membrane-Proximal Regions Identified By Computational Analysis

The possibility that structural similarities exist among viral envelope protein membrane-proximal cytoplasmic tail regions was addressed computationally. The hallmark characteristic of an amphiphilic structure is its hydrophobic moment (μ). Domains with a high μ value were calculated for a number of non-related viral envelope protein cytoplasmic sequences (Appendix 2, column C). For most viruses analyzed (one exception shown is influenza HA), the envelope protein cytoplasmic membrane-proximal region was calculated to have a high μ.

For comparison, Appendix 2 includes the C-terminal HIV-1 cytoplasmic tail segment 1 (μ value of 2.21) previously calculated to have the second highest μ value among all proteins in the data bank for 1989 (Eisenberg, et al., 1990). Segment 1 of the HIV-1 tail, however, is membrane-distal and is not essential for virus entry; it is included here to serve as an amphiphilicity reference. As in other viruses analyzed, a high μ also was identified for the HIV-1 envelope protein membrane-proximal cytoplasmic region. Because many lytic peptides are amphiphilic, it is relevant to note that the calculated envelope protein membrane-proximal μ values often are higher than those in lytic peptides. Also shown for comparison in Appendix 2 is the μ value of the lytic peptide melittin (1.23). The melittin fragment used in the cytoplasmic substitution has a μ value of 1.42.

In most of these high μ envelope protein regions, at least one amino acid is out-of-phase with respect to the amphiphilic character of the segment, reminiscent of Arg 609 in Moloney envelope protein (Appendix 2, column d). The possibility has been suggested that to cause efficient fusion a peptide must enter the membrane at an oblique angle (Martin et al., J. Virol., Vol. 70, pgs. 298-304 (1996)). A structural distortion by a helix-breaking proline (present in many helixes, Appendix 2, column d) or an out-of-phase polar residue may be involved in providing an oblique angle needed for membrane-destabilization during fusion. This observation may explain how Arg 609 contributes to membrane-destabilization.

In the analysis of different viral envelope protein sequences upstream to the high μ segment a recurrent proline was noticed. An adjacent cysteine and glycine are also common (not shown). Proline and glycine may provide flexibility between the membrane-spanning and the membrane-proximal helices. Proline can contribute to the formation of the L-shaped structure between two helices (Efimov, 1992). Cysteine, if lipid modified (demonstrated for HIV-1, SIV, RSV, MPMV, MoMuLV, some HA isolates), may serve as a protector against the disturbances at the tail reverberating into the ectodomain.

Thus, computational analysis predicts the presence of a high μ membrane-proximal domain in a number of viruses with features like those identified in MoMuLV. By analogy with the MoMuLV envelope protein, such a domain is suggested to be functional in viral entry.

Contribution Of The Envelope Protein Cytoplasmic Tail Region To Viral Incorporation

Several lines of evidence suggest that the viral cytoplasmic tail region of envelope protein interacts with core proteins. The incorporation of the retroviral envelope protein into virions appears to be selective (Suomalainen, et al., J. Virol., Vol. 68, pgs. 4879-4889 (1994)). An interaction of the cytoplasmic tail region with matrix protein has been suggested to be present in MPMV (Brody, et al., J. Virol., Vol. 68, pgs. 4620-4627 (1992)) and in HIV-1 (Freed, et al., 1996) as indicated by compensatory matrix and env mutants. Additionally, particle incorporation of SIV envelope protein was also suggested to be dependent on the envelope protein cytoplasmic domain (Vzorov, et al., 1996). This conclusion is further supported by the SIV matrix structure (Rao, et al., Nature, Vol. 378, pgs. 743-747 (1995)), the exposed side of which corresponds to the region affecting envelope protein incorporation. Current and previous (Januszeski et al., 1997) results from progressive cytoplasmic truncations of the MoMuLV envelope protein cytoplasmic tail region also suggest that efficiency of envelope protein particle incorporation correlates with the integrity of the cytoplasmic tail region of the envelope protein.

The specific interaction, however, between the envelope protein cytoplasmic tail region and matrix can be argued against due to the relative ease of pseudotyping viral particles with heterologous envelope protein containing short cytoplasmic tails (MuLV envelope protein and naturally truncated HIV-2 env) (Freed, et al., J. Virol., Vol. 69, pgs. 1984-1989 (1995)). But, according to the melittin-fragment substitution results, the presence of an amphiphilic α-helix in the envelope protein cytoplasmic tail region may be sufficient for efficient incorporation. Thus, the apparent paradox of pseudotyping may be explained by the conservation of the cytoplasmic membrane-proximal region's secondary structure.

A Hypothetical Model Of The Cytoplasmic Tail Architecture

(1). Data to be accounted for by a model. A hypothetical model is proposed below based on the following data. [1] The CD and EPR structural analysis indicates that the peptide representing region 598-616 forms a monomeric amphiphilic α-helix. The helix is embedded partially into the membrane and is oriented parallel to the lipid bilayer. [2] Peptide 598-616 has membrane destabilization activity demonstrated electrophysiologically. [3] The EPR and computer analysis suggest that Arg 609 is positioned to face the membrane bilayer. As tested by in vitro and in vivo assays, Arg 609 contributes to the membrane destabilization activity. [4] Progressive truncations of the region 598-616 correlate with a progressive decrease in envelope protein fusogenicity. [5] These envelope protein truncation mutants also exhibit a progressive loss of envelope protein incorporation and a progressive loss of titer. [6] Substitution of the heterologous amphiphilic α-helix from melittin for the envelope protein domain 598-616 results in formation of fully functional virions. [7] A high hydrophobic moment is calculated for a number of unrelated viral membrane-proximal regions, thereby suggesting that the secondary structure of the membrane-proximal domain may be a major determinant of its function.

(2). A hypothetical unit of the sub-cytoplasmic structure and its implication for envelope protein fusion. In the absence of structural data for the cytoplasmic tail region in the context of whole MoMuLV envelope protein any proposed conformation is highly speculative. The following model best fits the available data.

In FIG. 6A the membrane-proximal domain 598-616 is represented as connected flexibly to the membrane-spanning helix via Gly 595 and Pro 596. The CD data indicate that the peptide 598-616 is non-helical in the absence of a lipid-water interface, but currently no data is available on what the actual structure of the unprocessed cytoplasmic tail region may be. Taking into consideration the proximity of the membrane and of the other possible structure-organizing components (e.g., matrix) which may also influence the folding of the cytoplasmic tail region, the domain 598-616 is represented as a helix prior to R peptide cleavage. It is represented as a helix because the CD analysis of the peptide representing the whole cytoplasmic tail region (598-632) (data not shown) indicates that peptide 598-632 has a higher helical content in the presence of membrane vesicles than the peptide 598-616 alone.

To account for the increased envelope protein fusogenicity after R peptide cleavage in this model, the domain 598-616 is suggested to spiral up into the membrane, forming an amphiphilic 60 -helix parallel to the lipid bilayer. This burying of a helix is likely to create structural tension in between the two perpendicular helixes: the membrane-spanning (570-595) and the membrane-proximal (598-616). Such tension may translate into a membrane disturbance at the base of the membrane-spanning domain, as well as along the length of the now membrane-embedded helix 598-616. This suggested burying of the amphiphilic helix into the membrane with Arg 609 oriented towards the membrane is proposed to cause fusion-potentiating destabilization of the viral membrane inner leaflet.

One possible explanation for the observed fusogenicity of the envelope protein truncation mutant R 601*, and the loss of fusion in envelope protein mutants truncated one helical turn upstream of the 601, could be that the residues Gly 595, Pro 596 are not a part of a membrane-spanning α-helix, but instead form a perpendicular turn within the membrane. The potentially lipid-modified Cys 597 and the following first turn of an amphiphilic structure (residues 598-601) can already be expected to have a membrane-destabilizing activity. In fact, the structure of 601* env may have a similarity to the near-membrane base structure of the wild type envelope protein prior to R peptide cleavage. This suggestion would account for syncytia formation in the co-culture assay with cells that express env with the uncleaved R peptide. On the other hand, in case of the R-less envelope protein, the process of embedding trimeric amphiphilic α-helix 598-616 spiked by Arg 609 could be expected to form a membrane patch with a larger radius and force of lipid disturbance than in the case of 601* envelope protein or that of the uncleaved cytoplasmic tail envelope protein. This interpretation offers the basis for the significantly more aggressive syncytia formation by the 616* vs 601* envelope protein or the envelope protein with the uncleaved tail as seen from the data on the rates of fusion.

Whereas Arg 609 is required for the activity of the isolated peptide, as measured by the electrophysiologic assays, the presence of Arg 609 is not necessary, although it potentiates fusion in the context of the whole envelope protein. The data indicate that the truncations that eliminate Arg 609 or mutate it do not eliminate fusion, although they do reduce it from the maximum. A caveat to interpretation of data based on any cell-to-cell fusion assays is that the mechanism of syncytia formation may not be identical to virus-to-cell fusion. This caution also pertains to the correlation of the fusion and the transduction data obtained in different cell assays. The Arg 609 mutants and the other cytoplasmic tail region mutants are being analyzed further with the attention to the hypothesis of Martin et al., J. Virol., Vol. 70, pgs. 298-304 (1996) that a fusion peptide is active when it enters the membrane at an oblique angle.

The successful functional substitution with the melittin segment indicates the importance of the secondary structure of the domain 598-616 for its function. The melittin-like cytoplasmic domain may be argued to function not by substituting an analogous function, but merely by stabilizing the ectodomain; however, envelope protein chimeras with random and hydrophilic tails are not fusogenic. In addition, previous saturation mutagenesis data of the membrane-proximal region (Januszeski et al., 1997) indicate that mutations that disrupt amphiphilicity and reduce hydrophobicity of the membrane-proximal region have a negative effect on fusion. Thus, current data suggest that the structure of the membrane-proximal domain determines its function.

Although the scenario in which the envelope protein membrane-proximal region becomes a membrane-associating amphiphilic α-helix is hypothetical, it accounts for the current data. The proposed conformational change of the MoMuLV envelope protein cytoplasmic tail region is likely to occur during particle maturation, because it is concurrent with proteolysis of the R peptide. This process of membrane-destabilization by an insertion of an amphiphilic structure provides a possible explanation for how viruses may become primed for fusion. In combination with the subsequent action of the ectodomain's fusion peptide which becomes activated as a result of interaction with a host and inserts into the host bilayer, the destabilization of the viral membrane above the R-less tail may be sufficient to bring fusion of host and viral membranes to completion.

The hypothetical structure is modeled as a trimer (FIG. 6B) based on the crystallography of the MoMuLV ectodomain TM segment (Fass et al., 1996). Because during particle maturation, R peptide cleavage by the viral protease is inefficient, retaining more than 50% of the tails unprocessed, 1 out of 3 tails is shown as R-less. To account for the EPR measurements that suggest monomeric association with the membrane for peptides 598-632 (not shown) and 598-616, the cytoplasmic tail regions were modeled as monomers and then composed into a trimer. The relative position of the cytoplasmic tail regions as well as the angles between the membrane-spanning (573-594) and the membrane-proximal (598-616) segments constrained as α-helixes were generated by energy minimization using Quanta 4.0.

The current literature, as discussed, point to the possible interaction of the envelope protein cytoplasmic tail region with the viral core. The data on the cytoplasmic tail region truncations further indicate that the presence of the R peptide has a positive effect on incorporation, since its removal decreases envelope protein incorporation. Moreover, removing of the membrane-proximal region results in further significant decrease of envelope protein incorporation. Further, structurally solved retroviral ectodomain segments and matrices are both trimers, and the overall architecture of the three resolved viral matrices of HIV-1, SIV, and BLV (Hill, et al., Proc. Nat. Acad. Sci., Vol. 93, pgs. 3099-3104 (1996); Rao, et al., (1995); Matthews, et al., Embo J., Vol. 15, pgs. 3267-3274 (1996)) are reported to be very similar. From the data in Appendix 2, the argument can be made that the envelope protein membrane-proximal regions in a number of unrelated viruses may have a similar amphiphilic structure. In view of these data it is intriguing that the proposed MOMuLV cytoplasmic tail trimer (FIG. 6B) has an apparent architectural similarity to the upper surface of the crystallized matrices (Rao, et al., 1995; Hill, et al., 1995). The repetitive unit of the HIV-1 structure for matrix is shown in FIG. 6C for comparison. The surfaces of both trimers (the MoMuLV envelope protein cytoplasmic tail region and the lentiviral matrix) are outlined by three α-helixes forming an equilateral triangle with similar architecture and dimensions. The MoMuLV envelope protein sub-cytoplasmic trimer has a side of 67 Å and the SIV matrix measures at 68±8 Å (Rao, et al., 1995). At the corner of each membrane-parallel helix in the HIV-1 matrix crystal there is a long protruding helix which may serve as a support for the membrane-distal amphiphilic lentiviral tail. The proposed trimeric unit (FIG. 6B) can be arranged into a sub-membrane 2-D lattice of envelope protein cytoplasmic tails with architecture similar to that of the upper surface of the crystallized matrices (Rao, et al., 1995; Hill, et al., 1996). The possibility of matrix surface being congruent to the envelope protein sub-membrane structures has positive implications for viral assembly. These architectural correlations are amenable to further investigation into the possibility of matrix-envelope protein tail associations.

### Example 2

Peptides corresponding to the cytoplasmic tail region of the Moloney Murine Leukemia Virus envelope protein were synthesized according to the procedure described in Example 1. Unilamellar liposomes formed from (i) POPC; (ii) POPC and POPG at a molar ratio of POPC: POPG of 3:1, or (iii) POPC and POPG at a molar ratio of POPC: POPG of 1:1 were prepared in 100 mM KC1 according to the procedure described in Example 1. Peptide-induced K+ release from the liposomes also was detected according to the procedure described in Example 1. All measurements were carried out at 22° C. The results are given in Table III below.

TABLE III
% K⁺ - Release

| Peptide | POPC | POPC:POPG,3:1 | POPC:POPG1:1 |
|---|---|---|---|
| Triton X-100 | 100 | 100 | 100 |
| 602-616 | 2 | 4 | 5 |
| 617-632 | 0 | 0 | 0 |
| 598-616 | 9 | 13 | 24 |
| 598-611 | N/A | 13 | 22 |
| 598-616 V606C | 10 | 32 | 35 |
| 598-616 F605C | 8 | 4 | 7 |
| 598-616 Q604C | 29 | 20 | 28 |
| 598-616 D608C | 5 | 7 | 11 |
| 598-616 K607C | 29 | 37 | 49 |
| 598-632 Q604C | 6 | 30 | 31 |
| 598-616 V603C | 29 | 12 | 12 |
| 598-616 R601C | 32 | 10 | 13 |
| 598-616 L599C | 13 | 16 | 13 |
| 598-616 R609A | 11 | 0 | 0 |
| 617-632 L625C | 30 | 39 | 53 |
| 598-632 V606C | 0 | 14 | 17 |
| 598-616 R609V/ | 28 | 17 | 18 |
| V606R | | | |
| 597-616 | 11 | 11 | 15 |
| Magainin | 8 | 43 | 51 |
| Melittin | 31 | 31 | 27 |
| HIV segII V8C | 39 | 83 | 92 |

The disclosures of all patents, publications (including published patent applications), database accession numbers, and depository accession numbers referenced in this specification are specifically incorporated herein by reference in their entirety to the same extent as if each such individual patent, publication, database accession number, and depository accession number were specifically and individually indicated to be incorporated by reference.

It is to be understood, however, that the scope of the present invention is not to be limited to the specific embodiments described above. The invention may be practiced other than as particularly described and still be within the scope of the accompanying claims.

### APPENDIX 1

Effect Of Truncations, Mutations, And Substitutions In The Domain 598-616 On Cell Surface Expression, Fusion, Envelope Protein Incorporation, And Transduction.

A.

| Cytoplasmic | Cell Surface | Fusion | Particle | |
|---|---|---|---|---|
| Substitutions | Expression^{a} | NIH3T^{b} | Incorporation^{c} | Titer^{d} |
| A. | | | | |
| WT | 100 | 0 | ++++ | 100 |
| melittin fragment | 120+/−32 | 139+/−40 | +++ | 86+/−12 |
| hydrophilic | 69+/−33 | 1+/−1 | ++ | 0.05+/−0.04 |
| random | 40+/−3 | 8+/−4 | + | 0.04+/−0.004 |
| 616* | 95+/−6 | 100 | ++ | 38+/−23 |
| 601* | 58+/−5 | 23+/−3 | + | 6+/4 |
| | Cell Surface | Fusion | Particle | |
| Truncations | Expression^{a} | 293T/XC6^{b} | Incorporation^{c} | Titer^{d} |
| B. | | | | |
| WT | 100 | 100 | ++++ | 100 |
| 616* | 95+/−6 | 251+/−36 | ++ | 38+/−23 |
| 601* | 58+/−5 | 101+/−12 | + | 6+/−4 |
| 598* | 112+/−69 | 4+/−4 | +/− | 0.3+/−0.4 |
| 595* | 117+/−73 | 9+/−12 | +/− | 0.03+/−0.03 |
| 595SR* | 65+/−25 | 11+/−12 | +/− | 0.14+/−0.23 |
| 578* | 4+/−5 | 0+/−0 | − | 0+/−0 |
| GLA15E | 176+/−47 | 0+/−0 | +/− | 0+/−0 |
| ecto | | | | |
| | Cell Surface | Fusion | Particle | |
| Point Mutations | Expression^{a} | 293T/XC6^{b} | Incorporation^{c} | Titer^{d} |
| C. | | | | |
| WT | 100 | 100 | ++++ | 100 |
| R609C | 15+/−8 | 11+/−10 | − | 0.02+/−0.01 |
| R609A | 180+/−76 | 54+/−28 | ++++ | 65+/−37 |
| G595VP596A | 70+/−16 | 68+/−59 | ++++ | 43+/−39 |
| insertSR | 69+/−34 | 41+/−30 | ++++ | 85+/−21 |
| G595 | | | | |
| C597A | 81+/−30 | 57+/−7 | ++++ | 61+/−37 |
| C597S | 74+/−31 | 42+/−26 | ++++ | 41+/−33 |

^{a}The surface envelope protein expression measured by indirect immunofluorescense using env-specific antibody on env-transfected 293T cells.
^{b}The relative fusion efficiency was determined as syncytia formation in env-transfected NIH3T3 cells (Table A column^{b} only) or as the ability to induce syncytia formation in XC6 cell co-cultured with env-transfected 293T cells.
^{c}Qualitative representation of envelope protein levels in the Western Blots (n = 3-5) probed with antibodies against gag (p30), SU (gp70) and TM (p15E). Representative Western Blots are shown in FIG. 5.
^{d}A mutant ability to transduce host NIH3T3 cells is shown as relative β-gal titer. Same supernatant used for titers were used for Western Blots. The average wild type envelope protein titer was 4 × 10⁵ cfu/ml for (A) and 1 × 10⁶ cfu/ml for (B) and (C).
Abbreviations reflect the amino acid position of the mutation with the wild type residue in one letter code on the left and the mutant residue on the right. In the mutant SR 595 Ser and Arg are inserted before residue 595. For the truncation mutants an asterisk (*) represents a stop codon after the residue number indicated.

### APPENDIX 2

### Amphiphilicity evaluation of viral envelope protein

### membrane-proximal domains

| | membr- | | | | |
|---|---|---|---|---|---|
| | prox | | aa out of | | |
| | seg- | | helix | tail | notable MS |
| virus^{a} | ment^{b} | μ^{c} | phase^{d} | length^{e} | features^{f} |
| ALV | −2/14 | 1.83 | T12 | 30 | −4P,−3C |
| BLV | −2/17 | 1.67 | T4, P15 | 51 | −4P,−3C |
| EIA | 1/36 | 1.71** | P41, H45, | 52 | −18P,−17C, |
| | | | P49 | | −16C,−15G, |
| | | | | | −13P |
| FIV | −6/10 | 1.95 | T7 | 47 | −6C,−4P |
| HEP C | 1/17 | 1.36 | Q2, T6, P7 | 35 | −7P |
| HIV1 | −3/11 | 1.58 | T7 | 151 | −7R |
| HIV1 | | 2.21 | P17, R21, | 151 | NA |
| seg1*** | 128- | | G23 | | |
| | 151 | | | | |
| HIV2SYR | 1/25 | 1.39 | T10, P17 | 157 | −7R |
| HTLV2 | 1/12 | 1.87 | N2, R9 | 26 | −4P,−2C |
| hRSV | 1/21 | 0.9 | R3, P6, | 24 | −1C,−2Y |
| | | | S10, N13 | | |
| INF A1 | −2/11 | 0.7 | G5 | 11 | −2C |
| melittin | NA | 1.23 | P18 | 26 | NA |
| MoMuLV | −3/14 | 1.8 | R12 | 32 | −6G,−5P, |
| | | | | | −4C |
| MMTV | −6/13 | 1.6 | Q15 | 40 | −3C,−8P |
| MPMV | 1/22 | 1.26 | G3, Q17 | 46 | −6C, |
| | | | | | 3G,4P |
| RSV | −9/8 | 1.67 | S2, K5 | 40 | −9C, |
| | | | | | −7P,−6C |
| PINF | 1/17 | 1.11 | R10 | 35 | |
| SIV239 | −5/13 | 1.24 | R2 | 164 | −11R |
| SNV | −2/16 | 1.87 | K12 | 16 | −5G, |
| | | | | | −4P,−3C |
| VSV | −2/13 | 1.24 | G3, K12 | 29 | |
| SimSrcV- | −9/8 | 1.73 | R2 | 35 | −17G, 16P, |
| HLB | | | | | −4P |
| HSV gH | 1-17 | 1.45 | S6, Q17 | | |
| Marburg | 1-12 | 1.41 | Y10 | | |
| Measles F | 1-33 | 0.72 | NA | | |
| H | 2-24 | 0.36 | NA | | |
| Roto NSP4 | 9-31 | 1.55 | Q,Q,E | | |
| Semliki | 2 aa cy- | NA | NA | | |
| Forest | toplas- | 0.77 | NA | | |
| E1; | mic | | | | |
| E2 | tail; | | | | |
| | 3-11 | | | | |
| Sendi | 6-13 | 1.24 | K12 | | |
| Sindbis | 2 aa cy- | NA | NA | | |
| E1; | toplas- | 1.0 | NA | | |
| E2 | mic | | | | |
| | tail; | | | | |
| | 1-11 | | | | |
| SV5 | 1-18 | 1.06 | N14, R15, | | |
| | | | Q19 | | |
| ^{a}Virus name | | | | | |
| ^{b}Position of the calculated membrane-proximal amphiphilic region with respect to the 1st residue after the predicted membrane-spanning (MS) domain | | | | | |
| ^{c}Hydrophobic moment (μ) calculated using DNASIS software (Hitachi Software Engineering Co., Ltd.) employing the Chou, Fasman and Rose algorithm and calculated with the Kyte and Doolittle algorithm. | | | | | |
| ^{d}Hydrophilic residue on a hydrophobic side of the predicted amphiphilic region | | | | | |
| ^{e}The length of env cytoplasmic tail | | | | | |
| ^{f}Neighboring amino acids of notice (see text for discussion) | | | | | |
| *Abbreviations of viruses and proteins: avian leukosis - ALV, bovine leukemia - BLV, equine infectious anemia - EIA, feline immunideficiency - FIV, hepatitis C-HEPC, human immunodeficiency - HIV, human T cell leukemia - HTLV, human respiratory syncytial - hRSV, influenza - INF, murine mammary tumor - MMTV, Mason Pfizer monkey - MPMV, Rous Sarcoma - RSV, parainfluenza - PINF, spleen necrosis - SNV, vesicular stomatitis - VSV, simian sarcoma - Sim Src V-HLB, | | | | | |
| # Herpes Simplex virus glycoprotein H - HSV gH, Simian virus - SV5. | | | | | |

**calculated for the first 1-36 residues of a larger predicted amphiphilic structure
***HIV1 segments as defined by Eisenberg and Wesson (1990)
NA not applicable

1. An isolated peptide comprising a fragment of a viral envelope protein, wherein said peptide is free of the portion of the envelope protein N-terminal of the membrane-spanning region of the envelope protein, said peptide having a membrane-destabilizing activity.

2. The peptide of claim 1 wherein the membrane-destabilizing activity of said peptide is sufficient to induce an electrophysiologically detectable increase of the release of a suitable marker from a liposome at an active concentration of 30 mM peptide/1 mol lipid in a suitable assay.

3. The peptide of claim 2, wherein said peptide forms an α-helical amphiphilic structure.

4. The peptide of claim 3 having a hydrophobic moment μ of at least 0.9 as calculated using DNASIS software employing the Chou, Fasman and Rose algorithm and calculated with the Kyte and Doolittle algorithm.

5. The peptide of claim 4 wherein said fragment comprises at least 8 amino acids.

6. The peptide of claim 5 wherein said fragment comprises at least the first 8 amino acids of the N-terminal portion of the cytoplasmic tail region of the envelope protein.

7. The peptide of claim 6 wherein said fragment comprises at least one out-of-phase residue.

8. The peptide of claims 7 wherein a portion of said peptide is present in said membrane-spanning region of said viral envelope protein.

9. The peptide of claim 8 wherein said peptide comprises the amino acid sequence of SEQ ID NO: 1.

10. Derivatives and analogues of the peptide of claim 1 having at least one substitution of an amino acid residue that maintains the membrane-destabilizing activity of said peptide and/or having the reverse sequence of said peptide.

11. Use of the peptide of claim 1, or a nucleic acid encoding said peptide, for the preparation of a viral or synthetic vector.

12. Use of the peptide of claim 1, or a nucleic acid encoding said peptide, for the preparation of a medicament.

13. Use of an amphiphilic compound having a membrane-destabilizing activity for the preparation of a viral vector.

14. A peptide selected from the group consisting of (SEQ ID NO: 2) and (SEQ ID NO: 3) and derivatives and analogues of (SEQ ID NO: 2) and (SEQ ID NO: 3) having at least one amino acid substitution of (SEQ ID NO: 2) and (SEQ ID NO: 3) that maintains the membrane-destabilizing activity of said peptide.

15. The peptide of claim 14 wherein said peptide is (SEQ ID NO: 2).

16. The peptide of claim 14 wherein said peptide is (SEQ ID NO: 3).

17. A viral particle including a modified envelope protein, wherein said modified envelope protein includes the peptide of claim 1, wherein said peptide is located in a portion of said envelope protein external to the viral membrane.

18. The viral particle of claim 17 wherein said modified envelope protein further includes a targeting polypeptide including a binding region that binds to a liqand.

19. A retroviral vector particle including a retroviral envelope protein and the peptide of claim 1, wherein said peptide is attached to the retroviral membrane.

20. The retroviral vector particle of claim 19 wherein said retroviral envelope protein is a modified envelope protein that includes a targeting polypeptide that binds to a ligand.

21. A retroviral vector particle including a retroviral envelope protein, a targeting polypeptide including a binding region that binds to a ligand, and the peptide of claim 1, wherein each of said targeting polypeptide and the peptide of claim 1 is attached to the retroviral membrane.

22. A retroviral vector particle including a retroviral envelope protein, and a polypeptide including a targeting polypeptide including a binding region that binds to a ligand, a spacer moiety, and the peptide of claim 1, wherein said polypeptide is attached to the retroviral membrane.

23. A retroviral vector particle including (i) a targeting polypeptide including a binding region that binds to a ligand and (ii) the peptide of claim 1, wherein each of said targeting polypeptide and the peptide of claim 1 is attached separately to the membrane of said retroviral vector particle, and said retroviral vector particle does not include a retroviral envelope protein.

24. A retroviral vector particle including a polypeptide including (i) a targeting polypeptide including a binding region that binds to a ligand, (ii) a spacer moiety, and (iii) the peptide of claim 1, wherein said polypeptide is attached to the membrane of said retroviral vector particle, and said retroviral vector particle does not include a retroviral envelope protein.

25. The viral particle of claim 17 wherein said particle further includes at least one polynucleotide encoding a therapeutic agent.

26. The retroviral vector particle of claim 19 wherein said particle further includes at least one polynucleotide encoding a therapeutic agent.

27. The retroviral vector particle of claim 21 wherein said particle further includes at least one polynucleotide encoding a therapeutic agent.

28. The retroviral vector particle of claim 22 wherein said particle further includes at least one polynucleotide encoding a therapeutic agent.

29. The retroviral vector particle of claim 23 wherein said particle further includes at least one polynucleotide encoding a therapeutic agent.

30. The retroviral vector particle of claim 24 wherein said particle further includes at least one polynucleotide encoding a therapeutic agent.

31. A method of expressing a therapeutic agent in an animal, comprising: administering to an animal the viral particle of claim 25.

32. A method of expressing a therapeutic agent in an animal, comprising: administering to an animal the retroviral vector particle of claim 26.

33. A method of expressing a therapeutic agent in an animal, comprising: administering to an animal the retroviral vector particle of claim 27.

34. A method of expressing a therapeutic agent in an animal, comprising: administering to an animal the retroviral vector particle of claim 28.

35. A method of expressing a therapeutic agent in an animal, comprising: administering to an animal the retroviral vector particle of claim 29.

36. A method of expressing a therapeutic agent in an animal, comprising: administering to an animal the retroviral vector particle of claim 30.

37. A packaging cell including a polynucleotide encoding the retroviral gag protein, a polynucleotide encoding the retroviral pol protein, and a polynucleotide encoding a viral envelope protein including the peptide of claim 1.

38. The cell of claim 37 wherein said viral envelope protein further includes a targeting polypeptide including a binding region that binds to a ligand.

39. A producer cell formed from the packaging cell of claim 37.

40. A producer cell formed from the packaging cell of claim 38.

41. A packaging cell including a polynucleotide encoding the retroviral gag protein, a polynucleotide encoding the retroviral pol protein, a polynucleotide encoding the retroviral env protein, a polynucleotide including a nucleic acid sequence encoding the peptide of claim 1 and a nucleic acid sequence encoding a membrane-spanning region of a viral envelope protein, and a polynucleotide including a nucleic acid sequence encoding a targeting polypeptide including a binding region which binds to a ligand and a nucleic acid sequence encoding a membrane-spanning region of a viral envelope protein.

42. A producer cell formed from the packaging cell of claim 41.

43. A packaging cell including a polynucleotide encoding the retroviral gag protein, a polynucleotide encoding the retroviral pol protein, a polynucleotide encoding the retroviral env protein, and a polynucleotide including a first nucleic acid sequence encoding the peptide of claim 1, a second nucleic acid sequence encoding a spacer moiety, a third nucleic acid sequence encoding a targeting polypeptide including a binding region that binds to a ligand, and a fourth nucleic acid sequence encoding a membrane-spanning region of a viral envelope protein.

44. A producer cell formed from the packaging cell of claim 43.

45. A pre-packaging cell including a polynucleotide encoding the retroviral gag protein, a polynucleotide encoding the retroviral pol protein, a polynucleotide including a nucleic acid sequence encoding the peptide of claim 1 and a nucleic acid sequence encoding a membrane-spanning region of a viral envelope protein, and a polynucleotide including a nucleic acid sequence encoding a targeting polypeptide including a binding region that binds to a ligand and a nucleic acid sequence encoding a membrane-spanning region of a viral envelope protein.

46. A pre-packaging cell line including a polynucleotide encoding the retroviral gag protein, a polynucleotide encoding the retroviral pol protein, and a polynucleotide including (i) a first nucleic acid sequence encoding the peptide of claim 1, (ii) a second nucleic acid sequence encoding a spacer moiety, (iii) a third nucleic acid sequence encoding a targeting polypeptide including a binding region that binds to a ligand, and (iv) a fourth nucleic acid sequence encoding a membrane-spanning region of a viral envelope protein.
